(19) United States
(12) Patent Application Publication
(10) Pub. No.: US 2009/0104705 A1
(43) Pub. Date: Apr. 23, 2009

(54) Ascorbic acid conjugates

(76) Inventors: Jan Frederik Stevens, Corvallis, OR (US); John D. Sowell, Corvallis, OR (US); Balz Frei, Corvallis, OR (US)

Correspondence Address: KLARQUIST SPARKMAN, LLP, 121 SW SALMON STREET, SUITE 1600, PORTLAND, OR, 97204, US

(21) Appl. No.: 11/920,498

(22) PCT Filed: May 23, 2006
(86) PCT No.: PCT/US06/19890

§ 371 Date: Nov. 16, 2007

Related U.S. Application Data
(60) Provisional application No. 60/683,929, filed on May 23, 2005.

(51) Int. Cl. G01N 33/00

(52) U.S. Cl. 436/63; 436/129; 436/93

(57) ABSTRACT

Oxidative stress, resulting from the generation of reactive oxygen species, contributes to the development of a multitude of age-related diseases. Current methods of assessing oxidative stress levels range from the detection of lipid peroxidation products, such as F₂-isoprostanes and malondialdehyde, to monitoring the redox status of glutathione. While useful, traditional biomarkers of oxidative stress are not without their drawbacks, including low in vitro concentrations and possible artifact formation. Disclosed herein are new marker compounds, including ascorbylated 4-hydroxy-2-nonenal, that are useful as biomarkers of oxidative stress.

### CROSS REFERENCE TO RELATED APPLICATION

This application claims the benefit of the earlier filing date of U.S. provisional patent application No. 60/683,929 filed May 23, 2005, which is incorporated herein by reference.

### ACKNOWLEDGEMENT OF GOVERNMENT SUPPORT

This invention was made with government support under HL60886 awarded by the National Institutes of Health. The government has certain rights in the invention.

### FIELD

Disclosed herein is a method for assessing risk of oxidative stress-related disorders, such as atherosclerosis.

### BACKGROUND

Coronary heart disease is the single leading cause of death in the United States, while stroke ranks third after cancer (American Heart Association, 2004). Atherosclerosis is usually the underlying vascular disease. It is generally accepted that lipid peroxidation, oxidation of low-density lipoprotein (LDL) and endothelial activation play central roles in atherogenesis (Diaz et al, 1997; Steinberg and Witztum 2002). Lipid peroxidation is a radical chain reaction, initiated by reactive oxygen species (ROS), that can be inhibited by scavengers of ROS, notably vitamins C and E. Recent work, however, indicates that vitamin C is capable of degrading lipid hydroperoxides that subsequently form cytotoxic and genotoxic α,β-unsaturated aldehydes. These seemingly paradoxical roles of vitamin C suggest that its biological interactions are likely to be far more complex than previously thought.

LPO processes contribute to the chronic inflammatory component typical of many age-related diseases. Examples are diabetes, atherosclerosis and auto-immune diseases like lupus erythematosus (SLE). Recent Japanese findings indicate that vitamin C intake is inversely correlated with the risk of active SLE and suggest that vitamin C intake is inversely correlated with the risk of active SLE and suggest that vitamin C supplementation may prevent the onset of active SLE (Minami et al. 2003). There is little doubt that the antioxidant properties of ascorbic acid contribute to an overall anti-inflammatory effect through scavenging reactive oxygen species. It becomes more difficult to predict the net result of vitamin C's direct interactions with oxidized lipids on disease development and progression.

Oxidative stress has been linked to a multitude of diseases, including atherosclerosis, Alzheimer's disease and autoimmune disorders such as lupus and rheumatoid arthritis. Consequently, tools for the assessment of cellular oxidative stress levels are of interest. Current strategies for assessing oxidative stress levels range from the detection of lipid peroxidation products, such as F₂ isoprostanes, 4-hydroxy-2-nonenal and malondialdehyde, to monitoring the redox status of antioxidant compounds. While these approaches are useful, chemical instability artifact formation are potential concerns. Disclosed herein is a method for monitoring oxidative stress in a subject, using lipid peroxidation product conjugate compounds, such as an ascorbyl-HNE conjugate, as novel biomarkers of oxidative stress.

### SUMMARY

Disclosed herein is a diagnostic method for evaluating the likelihood that a subject has or is at risk of an oxidative stress related disorder. Examples of such disorders include, without limitation, Alzheimer's disease and autoimmune disorders such as lupus and rheumatoid arthritis and cardiovascular disease, such as atherosclerosis.

In one embodiment the method includes detecting a concentration of an ascorbic acid-lipid peroxidation product conjugate. The conjugate typically is formed from a lipid peroxidation product, such as an aldehyde or other reactive electrophile and ascorbic acid. In one embodiment, the conjugates comprise a lipid peroxidation product derived from linoleic acid, such as a 4-hydroxy-2-nonenal residue.

In a further embodiment of the method, a concentration of an ascorbic acid-lipid peroxidation product conjugate is correlated with a second biomarker involved in inflammation, such as sVCAM-1, sICAM-1, E-selectin and/or MCP-1.

Also disclosed herein are examples of a kit for detecting an ascorbic acid-lipid peroxidation product conjugate. Such kits can be used to identify or evaluate subjects for the existence or presence of oxidative stress-related disorders. Such kits include an amount of an ascorbic acid-lipid peroxidation product conjugate (e.g., in the form of a pharmaceutical composition) and optionally include a reference standard for quantitative analysis. The kit may further include instructions for using the kit for its intended purpose(s).

The foregoing and other objects, features, and advantages of the invention will become more apparent from the following detailed description, which proceeds with reference to the accompanying figures.

### BRIEF DESCRIPTION OF THE DRAWINGS

FIG. 1 illustrates the formation of the ascorbyl-HNE conjugate wherein ascorbic acid acting as a nucleophile forms a conjugate with 4-hydroxy-2-nonenal via Michael addition chemistry.

FIG. 2 includes tandem mass spectrometry analysis of the ascorbylated-HNE. MS/MS daughter scans of the m/z 350 [M+NH₄]⁺ ion for the (A) synthetic ascorbyl-HNE adduct and (B) the ascorbyl-HNE conjugate in human plasma.

FIG. 3 includes liquid chromatography-tandem mass spectrometry analyses with multiple reaction-monitoring of plasma for the presence of ascorbylated 4-hydroxy-2-nonenal.

FIG. 4 includes calibration curves for the ascorbyl-HNE adduct, wherein analyte/internal standard response ratios were plotted as a function of analyte concentration; ascorbylated 2-octenal was used as the internal standard.

FIG. 5A includes chromatograms of the ascorbyl-HNE conjugate and internal standard for nonsmokers.

FIG. 5B includes chromatograms of the ascorbyl-HNE conjugate and internal standard for smokers.

FIG. 6A is a standard addition experiment for nonsmokers wherein curve A1 was derived from the analysis of aliquots of nonsmoker plasma sample, spiked with various concentrations of synthetic ascorbyl-HNE adduct and a fixed amount of internal standard, and curve A2 represents an external calibration curve derived from analysis of various concentrations of the ascorbyl-HNE adduct with a fixed concentration of internal standard

FIG. 6B is a standard addition experiment for smokers wherein curve B1 was derived from the analysis of aliquots of a smoker plasma sample, spiked with various concentrations of synthetic ascorbyl-HNE adduct and a fixed amount of internal standard, and curve B2 represents an external calibration curve derived from analysis of various concentrations of the ascorbyl-HNE adduct with a fixed concentration of internal standard.

FIG. 7A is a graph of plasma levels of F_{2α}-isoprostane in smokers (n=10) and nonsmokers (n=10), presented as mean±SE.

FIG. 7B is a graph of plasma levels of the ascorbyl-HNE conjugate in smokers (n=10) and nonsmokers (n=10), presented as mean±SE. LC-MS/MS injections were done in duplicate and averaged for all subjects. Results in both panels are statistically significant at the p<0.05 level.

FIG. 8 includes scatter and box plots of F2-isoprostane and ascorbyl-HNE levels in smokers (S, n=10) and nonsmokers (NS, n=10).

FIG. 9 is a scatter plot of ascorbic acid and ascorbyl-HNE conjugate plasma levels in smokers (n=10) and nonsmokers (n=10) measured by HPLC with amperometric detection.

FIG. 10 is an ORTFP representation of the X-ray structure of ascorbylated acrolein mono-hydrate.

FIG. 11 illustrates the results of LC-MS/MS analysis of the reaction between ascorbic acid and HPODE at different ascorbic acid/HPODE ratios.

FIG. 12 shows the LC/MS/MS analysis of an isotopomeric mixture of ¹²C and ¹³C₆-asorbylated HNE.

FIG. 13 illustrates the effect of HNE and ascorbylated HNE on the viability of HAECs as measured by the MTT assay.

FIG. 14 is a bar graph illustrating the effect of HPODE (35 μM) and ascorbate-treated HPODE (35 μM) on ICAM-1 expression in HAECs, expressed as mean of five observations (±SD).

FIG. 15 is a liquid chromatography-tandem mass spectrometry (multiple-reaction monitoring) analysis of a plasma sample from a 38 year-old male demonstrating the presence of additional ascorbyl-LPO product conjugates.

FIG. 16 is a bar graph charting plasma levels of ascorbyl-HNE conjugate in patients with established CAD (n=6) and in age-matched control subjects (n=7), presented as mean±SD.

### DETAILED DESCRIPTION

Disclosed herein are methods and reagents for assessing oxidative stress and related disorders using novel biomarkers disclosed herein. In general the biomarkers disclosed herein are conjugates formed from ascorbic acid and a lipid peroxidation product.

The following explanations of terms and methods are provided to better describe the present compounds, compositions and methods, and to guide those of ordinary skill in the art in the practice of the present disclosure. It is also to be understood that the terminology used in the disclosure is for the purpose of describing particular embodiments and examples only and is not intended to be limiting.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

In this specification and in the claims which follow, reference will be made to a number of terms which shall be understood to have the following meanings:

“Optional” or “optionally” means that the subsequently described event or circumstance can but need not occur, and that the description includes instances where said event or circumstance occurs and instances where it does not.

The term “antibody” means an immunoglobulin, whether natural or wholly or partially synthetically produced. All derivatives thereof which maintain specific binding ability are also included in the term. The term also covers any protein having a binding domain which is homologous or largely homologous to an immunoglobulin binding domain. These proteins may be derived from natural sources, or partly or wholly synthetically produced. Antibodies used herein may be monoclonal or polyclonal.

The term “antibody fragment” refers to any derivative of an antibody which is less than full-length. In an exemplary embodiment, the antibody fragment retains at least a significant portion of the full-length antibody's specific binding ability. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

“Atherosclerosis” refers to the progressive narrowing and hardening of a blood vessel over time. Atherosclerosis is a common form of arteriosclerosis in which deposits of yellowish plaques (atheromas) containing cholesterol, lipoid material, and lipophages are formed within the intima and inner media of large and medium-sized arteries. Coronary artery disease (CAD) describes is manifested, for example, in subjects having had myocardial infarction, coronary artery bypass graft surgery, percutaneous coronary intervention, a stenosis of 50% or greater in one or more major coronary vessels on angiography or additional peripheral arterial.

An “analyte” is a compound subject to analysis. In one embodiment, the compound is a biomarker of oxidative stress, such as an ascorbic acid-lipid peroxidation product conjugate

The term “biomarker of oxidative stress” refers to a compound, protein or reaction product that can be observed to increase or decrease in concentration in response to and/or coincident with oxidative stress. One example of such a biomarker described herein is an ascorbic acid-lipid peroxidation product conjugate.

The term “control value” as used herein refers to a basal level of biomarker, such as an ascorbic acid-lipid peroxidation product conjugate, that is normal, an amount present in a corresponding healthy cohort in the absence of any pathology (disease or disorder) associated with oxidative stress. Disclosed herein are methods and compositions for determining control values for oxidative stress. Such control values may need to account for age of the individual and therefore be directed to certain age ranges, as oxidative stress may accumulate over time. Such control values may additionally account for gender and race, and for environmental exposures, e.g., smoking, diet, etc.

“Derivative” refers to a compound or portion of a compound that is derived from or is theoretically derivable from a parent compound.

The term “detection” as used herein means determination that a substance, for example, a biomarker, such as an ascorbic acid-lipid peroxidation product conjugate is present. The methods and compositions of this invention also can be used to quantify the amount of or concentration of a substance, for example, biomarker, in a sample. Quantification and detection of biomarkers can be performed by any means known to those skilled in the art. In one embodiment, a biomarker is detected and/or quantified using mass spectrometry. Other means of detection and quantification include, without limitation, detection of the biomarker by an antibody which binds to the biomarker.

Biomarkers can be detected and quantified in samples including, but not limited to, plasma, serum, cerebrospinal fluid, saliva, semen, pleural fluid, peritoneal fluid and amniotic fluid samples. These samples may be of human origin or they may be taken from animals other than humans, for example, avian species, but preferably mammals. As will be apparent to those skilled in the art, the subject methods can be used to detect and quantify an ascorbic acid-lipid peroxidation product conjugate in non-biological samples.

The term “oxidative stress” as used herein refers to damage to biological molecules resulting from oxidation. Examples include but are not limited to oxidation of lipoproteins, membrane phospholipids; lipid peroxidation; protein damage, including cleavage of amino acid bonds and oxidation of functional groups; nucleic acid strand breaks; nucleic acid base modifications leading to point mutations; inhibition of RNA and protein synthesis; protein cross-linking; impaired maintenance of membrane ion gradients; and depletion of cellular levels of ATP, leading to cellular dysfunction and eventually to disease. The oxidant (oxidizing reagent) can be endogenous or exogenous.

Provided herein is a method for detecting and measuring accumulated oxidative stress. The term “accumulated oxidative stress” refers to oxidative stress which is present in a subject at the time of detection and measurement; such damage has not been repaired or otherwise removed.

The term “subject” includes both human and veterinary subjects.

Subjects at risk for an oxidative stress related disorder include those having inflammatory disorders, autoimmune disorders, such as rheumatoid arthritis and lupus and neurodegenerative disease, especially Alzheimer's disease or amyotrophic lateral sclerosis (ALS). More specifically, subjects at risk for such disorders may have a condition, such as atherosclerosis, cerebral ischemia, hepatopathy, diabetes, nervous diseases, renal diseases, hepatic cirrhosis, arthritis, retinopathy of prematurity, ocular uveitis, retinal rust disease, senile cataract, asbestos diseases, bronchial failures due to smoking, cerebral edema, pulmonary edema, foot edema, cerebral infarction, coronary artery disease, hemolytic anemia, progeria, epilepsy, Crohn's disease, Kawasaki disease, collagen disease, progressive systemic sclerosis, herpetic dermatitis, immune deficiency syndrome or the like.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise.

Although methods and materials similar or equivalent to those described herein can be used in the practice or testing of this disclosure, suitable methods and materials are described below. The term “comprises” means “includes.”

All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### I. INTRODUCTION

Chronic inflammation and oxidative stress are associated with a wide variety of diseases and disorders in human populations. Such diseases and disorders affect organs and systems including, but are not limited to, reproductive organs, immune system, lungs, cardiovascular system, nervous system, gastrointestinal system, as well as organs and systems controlling growth and development. Such diseases include, but are not limited to, coronary artery disease, renal disease, cancer, and psychiatric diseases. Disclosed herein are methods and reagents for assessing oxidative stress. Embodiments of the methods are useful for identifying a subject at risk of disorders, such as those listed above. Early identification of this risk could aid therapeutic intervention that ameliorates a sign or symptom of a nascent disease or pathological condition.

Polyunsaturated fatty acids (R₁—CH═CH—CH₂—CH═CH—R₂ or LH) are sensitive to reactions with ROS because abstraction of allylic hydrogens in these lipids by oxygen radicals leads to stabilized pentadienyl radicals (R₁—CH═CH—.CH—CH═CH—R₂ or L.) that spontaneously react with molecular oxygen to yield lipid peroxides (LOOHs).

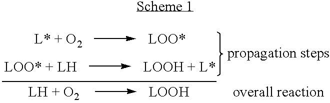

Oxidation of linoleic acid (18:2) occurs at the allylic positions 9 and 13, forming the hydroperoxy octadecadienoic acids, 9- and 13-HPODE. Arachidonic:acid (20:4) yields six hydroperoxy eico-satetraenoic acids, i.e., 5-, 8-, 9-, 11-, 12- and 15-HPETE, by oxygenation. In tissues, these lipid peroxides are predominantly reduced to hydroxyl acids (LOHs), by, for example glutathione peroxidase. However, a fraction of lipid peroxides are converted into LO. radicals that undergo α,β-carbon-carbon bond cleavage (β-scission), thereby generating α,β-unsaturated aldehydes. Additional secondary LPO products are formed by retro-aldol and epoxidation reactions. Examples of LPO products derived from 9- and 13-HPODE are illustrated in Scheme 2.

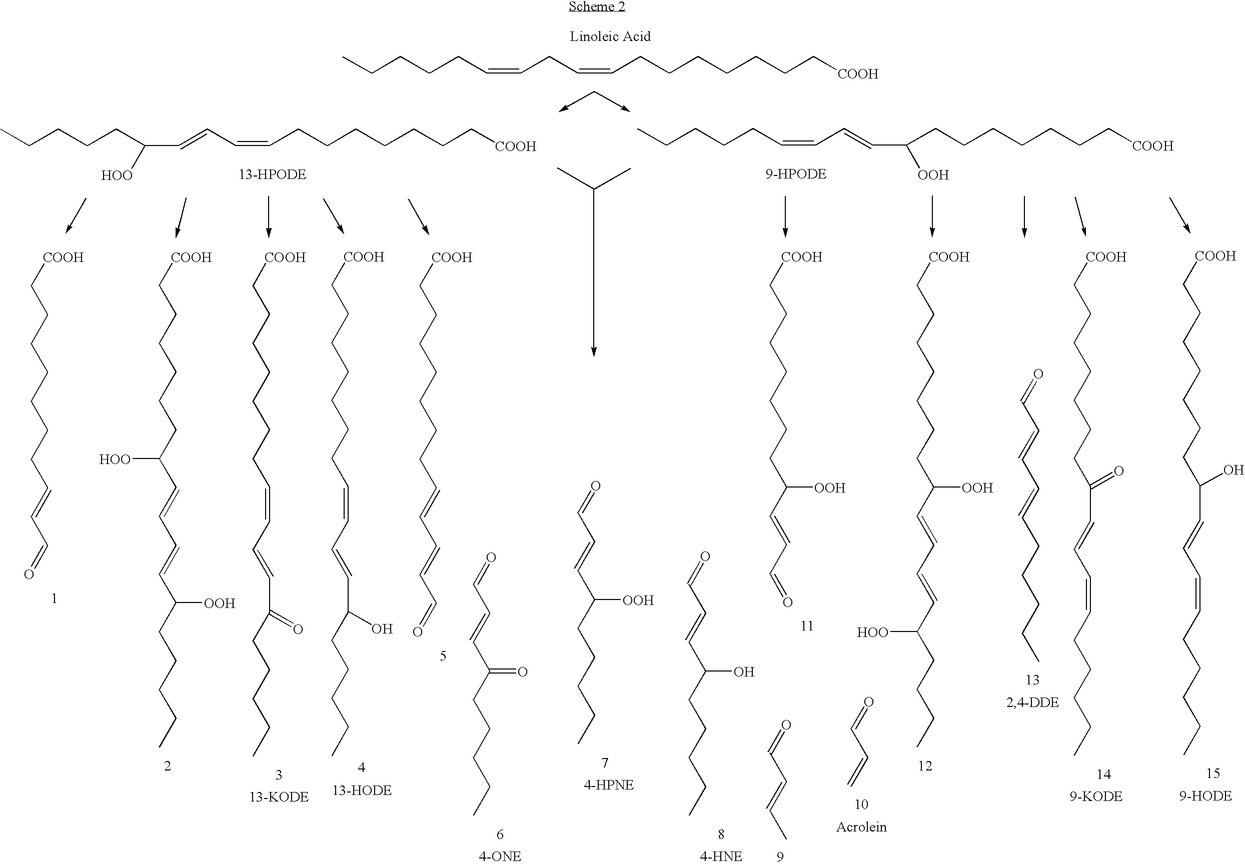

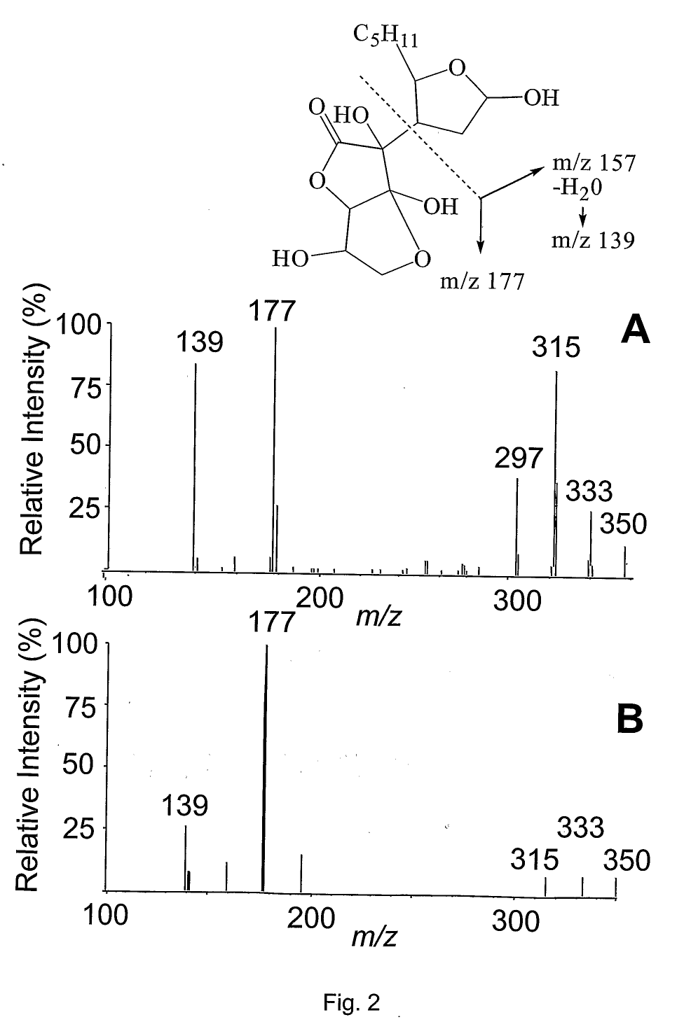

With reference to Scheme 2, a variety of lipid peroxidation products derived from 9- and 13-HPODE are known. With continued reference to Scheme 2, lipids 1, 3, 5, 6-11, 13 and 14 contain Michael acceptor systems. Such Michael acceptor systems can react with ascorbic acid to form ascorbic acid-lipid peroxidation product conjugates. Described herein are the detection of the Michael addition products of lipid hydroxyl acids 6, 8, and 10 with ascorbic acid. Also described herein is the conjugate formed from the reduced lipid hydroxyl acid analog of 11. These ascorbyl conjugate compounds have been detected both in vitro and in vivo (see FIGS. 2, 3 and 12).

Scheme 3 below, provides an overview of certain aspects of the relationship between oxidative stress, an ascorbic acid-lipid peroxidation product conjugate and disease pathogenesis in atherosclerosis.

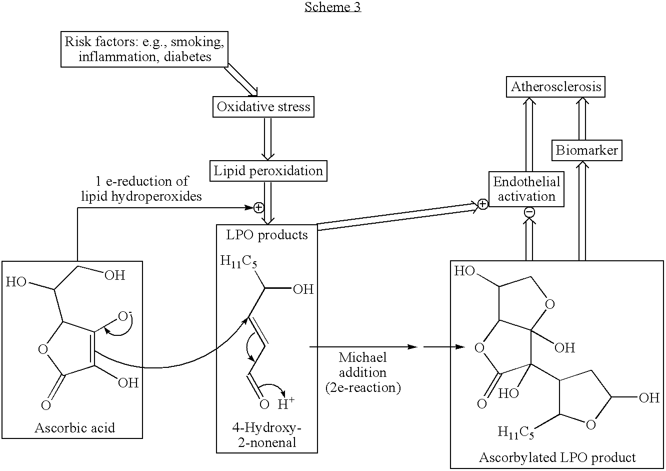

### II. LIPID PEROXIDATION PRODUCTS

In principle, conjugates of ascorbic acid with any electrophilic lipid peroxidation product can be used as in embodiments of the disclosed methods. In particular, LPO products having a Michael acceptor moiety, such as an alpha-beta unsaturated aldehyde or ketone group, are particularly suitable. In addition, certain LPO products can be correlated with one or more disease states. As such, conjugates of these products are of particular interest for monitoring as disclosed herein. For example, linoleic acid-derived LPO products, notably 4-hydroxynonenal and 2,4-decadienal, have been detected in oxidized low-density lipoprotein (ox-LDL). Additionally, bioassay-guided fractionation of lipids from mildly-oxidized LDL has provided evidence that most of the atherogenic activity, e.g., monocyte binding to endothelial cells, can be attributed to oxidized derivatives of arachidonic acid in phospholipid-bound form. Accordingly, methods for monitoring conjugates of both linoleic acid and arachidonic acid peroxidation products are disclosed herein.

Oxidative modification of arachidonic acid esters, yielding pro-inflammatory and atherogenic phospholipids, appears to be mediated primarily by 12-lipoxygenase in endothelial cells, but can also proceed non-enzymatically by interaction with reactive oxygen species or by direct reaction with HPODE to form HPETE derivatives. Numerous (78) LPO products derived from arachidonic acid have been reported. No less than 36 of these LPO products contain α,β-unsaturated carbonyl functionalities (Michael acceptor systems) or other electrophilic moieties (e.g., epoxide groups), that may form covalent adducts with ascorbic acid. Additional electrophilic oxylipids may be formed via the isoprostane pathway. A prominent example is the phospholipid-bound 5,6-epoxyisoprostane E₂, which accounts for more than 80% of the ox-LDL-induced monocyte chemotactic activity of endothelial cells. Although initial oxidative modification of lipids most likely takes place in esterified polyunsaturated fatty acids, oxidized derivatives of fatty acids may be released from bound forms by phospholipase A₂, which is activated as a response to inflammation and oxidative stress in monocytes and endothelial cells. Suitable lipid peroxidation products that can be monitored via detection of their ascorbyl conjugates are described for example in Spiteller et al. Aldehydic lipid peroxidation products derived from linoleic acid. Biochimica et Biophysica Acta 2001 1531 188-208; which is incorporated herein by reference.

Oxidative stress and inflammation are closely associated with endothelial activation and atherogenesis. Evidence suggests that NADPH oxidase-derived superoxide (O₂.⁻), myeloperoxidase-derived hypochlorous acid (HOCl) and peroxynitrite (O₂.⁻+NO.→ONOO⁻) are the key oxidants responsible for triggering a complex chain of events leading to atherosclerosis. These oxidants may directly interact with components of inflammatory signaling pathways or via LPO products formed as discussed above. Such interactions lead to activation of redox-sensitive transcription factors, notably nuclear factor κB (NFκB) and activator protein-1 (AP-1), which, in turn, increase the expression of inflammatory cytokines, monocyte chemoattractant protein-1 (MCP-1) (Valente et al. 1992), and the cellular adhesion molecules, vascular cell adhesion molecule-1 (VCAM-1), intercellular adhesion molecule-1 (ICAM-1) and E-selectin (Albelda et al. 1994; Lum and Roebuck 2001). Adhesion molecules are members of the immunoglobulin superfamily that, together with MCP-1, mediate monocyte recruitment from the circulation to the vascular wall, a critical early atherogenic event. The production of inflammatory cytokines, such as tumor necrosis factor α (TNFα) and interleukin-1β (IL-1β), further enhances the inflammatory responses of endothelial cells and other vascular cells, leading to a chronic inflammatory state of the vascular wall. There is increasing evidence that cytokines and other receptor ligands are capable of stimulating NADPH oxidase isoforms, e.g., NOX4 in endothelial cells, which may promote LPO processes through intracellular production of O₂.⁻ and H₂O₂.

Another LPO product is 4-hydroxy-2-nonenal (HNE), a known constituent of ox-LDL that is formed from both linoleic and arachidonic acid. HNE was shown to dose-dependently and reversibly inhibit IL-1β-stimulated NFκB activation in human monocytic cells, suggesting an anti-atherogenic role for HNE. The inhibitory effect of HNE on NFκB activation was attributed to the prevention of phosphorylation of IκB-α, an inhibitory subunit of NFκB. Moreover, exposure of HUVECs to HNE led to inhibition of NFκB activation and ICAM-1 expression (Herbst et al. 1999). By contrast, 13-HPODE, a source of HNE, has been shown to enhance ICAM-1 expression in HUVECs in the presence or absence of the cytokines, TNFα and IL-1β. The reduction product of 13-HPODE, 13-HODE, was a more potent inducer of ICAM-1 in the same study. Similar to the observations made with HNE, inhibition of E-selectin expression was observed when HAECs were exposed to the γ-hydroxy alkenal, 8-oxo-5-hydroxy-6-octenoic acid in phospholipid-bound form. These data suggest that LPO products interact in different ways with mediators of inflammation, as pointed out by. As disclosed herein, HPODEs are readily degraded and that their degradation products are conjugated by vitamin C. However, in vitro experiments including exposure of endothelial cells to LPO products may not adequately reflect the in vivo situation because cultured endothelial cells are usually vitamin C-deficient.

There is little doubt that oxidative stress is a key factor in the pathogenesis of atherosclerosis as stated by the oxidative modification hypothesis of atherosclerosis. See, Steinberg, D., Parthasarathy, S., Carew, T. E., Khoo, J. C., and Witztum, J. L. Beyond cholesterol. Modifications of low-density lipoprotein that increase its atherogenicity. N Engl J Med 1989, 320 915-924. And yet vitamin C and E supplementation shows no significant beneficial effect against cardiovascular disease in large-scale, double-blind, placebo-controlled trials. Briefly, three reasons for this apparent discrepancy are discussed below.

First, most trials are designed to last for a limited number of years with patients already having established cardiovascular disease (secondary prevention). However, it is possible that vitamin C offers protection against the early, pre-clinical stages of atherosclerosis (primary prevention), possibly by ascorbylation of inflammatory LPO products that would otherwise induce inflammatory responses or oxidatively modify LDL via Michael chemistry involving lysine and histidine residues in apo-lipoproteins. Accordingly, the reduction of ICAM-1 expression observed in normal human subjects after vitamin C supplementation could be explained by LPO product ascorbylation as a pathway for elimination of inflammatory LPO products.

Second, prior to the present disclosure, there was a lack of reliable biomarkers that could be used to identify patients at high risk for cardiovascular disease as a result of oxidative stress. Such patients would benefit most from supplementation with antioxidant vitamins. By contrast, those patients that are not under oxidative stress would not benefit from antioxidant treatment, which would ‘dilute’ the study population and diminish the study's power to detect a significant effect. While plasma cholesterol measurements are widely performed, they are not likely to be good indicators of oxidative stress. Other metabolites like isoprostanes and nitrotyrosine have much greater value as oxidative stress markers for cardiovascular disease. Thus, the disclosed ascorbylated LPO products serve the critical need for reliable biomarkers of oxidative stress.

Third, most supplementation trials were designed to measure clinical endpoints, without testing the efficacy of the antioxidant vitamin by using a relevant marker. Without having an independent marker to show an antioxidant effect of vitamin C or E supplementation, the outcome of such trials cannot be properly evaluated. Again, examples of ascorbylated LPO products and embodiments of the method for use thereof disclosed herein meet this important need.

### III. EXAMPLES

### General Materials and Methods

HPLC-grade acetonitrile was from Burdick and Jackson (Morristown, N.J.). All other chemicals were obtained through Sigma Chemical (St. Louis, Mo.).

All HPLC experiments were performed using a C18 column (250×1 mm, 4 μm, Synergi Max RP; Phenomenex, Torrance, Calif.) with a flow rate of 50 μl/min. The column was interfaced directly to the mass spectrometer. Solvent A was 10 mM ammonium acetate and 0.1% (v/v) formic acid in MilliQ water (pH 4.0). Ammonium acetate was added to the solvent to aid in the ionization process, as not all species analyzed were efficiently protonated. Solvent B was acetonitrile. A linear gradient, 25% B to 85% B over 45 min, was used.

Mass spectrometry experiments were conducted on a Perkin-Elmer Sciex API m Plus triple quadrupole mass spectrometer, operated in positive ion mode and equipped with an electrospray ion source (Concord, Canada). Nitrogen was used as the curtain gas and zero air was used as the sheath gas. For collision-induced dissociation experiments, argon was used as the collision gas, with a collision energy of 15 eV. For product ion and precursor ion scanning, a scan rate of 2 seconds was used.

Plasma samples were obtained from subjects who participated in a recently completed study at the Linus Pauling Institute. Participants were recruited on the basis of normal lipid status (total cholesterol <200 mg/dL; triglycerides <200 mg/dL), age (18-35 years), non-nutritional supplement use for greater than six months, and exercise status (<5 h/w of aerobic activity). Smokers were selected if they smoked >10 cigarettes/d, and smoking status of participants was verified by the measurement of urinary cotinine (Diagnostics Products Corp, CA). As suggested by the manufacturer, a urinary cotinine concentration of >500 ng/mL was used as a cutoff to confirm smoking status.

A blood sample was obtained from the antecubital vein of each participant after an overnight fast (˜12 h) into blood collection tubes (Vacutainer, Becton Dickinson, Franklin Lakes, N.J.) containing sodium heparin. Smokers were asked to refrain from smoking for 1 h prior to blood collection to obviate transient oxidative stress effects. Plasma was separated by centrifugation (500×g, 15 min, 4° C.; Beckman TJ-6, Palo Alto, Calif.), aliquoted into cryovials, snap frozen in liquid nitrogen, and then stored at −80° C. until analysis. Urine was collected for 24 h on a single occasion to evaluate urinary cotinine. Aliquots of urine were stored at −80° C. until analysis.

Two hundred-μl aliqots of human plasma were acidified with 250 μl of 0.1 M HCl. One ml of water was then added. Ethyl acetate was used to extract the ascorbyl-HNE conjugate (3×3 ml). The combined organic layers were then dried under a stream of nitrogen. The residue was redissolved in 65 μl of ethanol and mixed with 65 μl of LC Solvent A. Prior to injection, the samples were centrifuged for 5 min at 10,000 RPM. Injection volumes were 20 μl. Controls for the formation of ex vivo-artifact formation were performed as described previously [40].

A curve allowing for the determination of ascorbylated HNE in human plasma was constructed utilizing liquid chromatography with tandem mass spectrometry operated in multiple reaction monitoring mode (LC/MS/MS-MRM). Varying amounts of the ascorbyl-HNE adduct were mixed with a fixed amount of the internal standard, ascorbyl-octenal, to give 0.5, 1.0, 5.0, 10 and 50 μM of the analyte and 25 μM of the internal standard. Analyte/internal standard response was plotted against analyte concentration. The transitions m/z 350→m/z 177 and m/z 320→m/z 223 were used for quantitation of the synthetic standard and internal standard, respectively. Injections were done in triplicate, with 20 μL injection volumes.

Plasma samples from twenty subjects, ten smokers and ten nonsmokers, were prepared as described above, with the exception that the samples were spiked with internal standard (ascorbyl-octenal, 25 μM final concentration) prior to extraction. The samples were analyzed by LC/MS/MS-MRM and the concentration of the ascorbyl-HNE adduct determined through comparison with a calibration curve. Injections were done in duplicate, using 20 μL-injection volumes.

Statistical analysis was performed using GraphPad Prism (version 4.0) obtained from GraphPad Software (San Diego, Calif.). An unpaired Student's t-test was used for all comparisons between smokers and nonsmokers. Data were considered statistically significant if p <0.05. All data are reported as mean±SD unless otherwise noted.

Two hundred-μl-Aliquots of a plasma sample were spiked with varying amounts of ascorbyl-HNE adduct to give concentrations of 1.0, 2.0, 4.0 and 6.0 μM. The plasma samples were also spiked with internal standard (ascorbyl-octenal, 25 μM final concentration). The samples were analyzed by LC/MS/MS-MRM as described above.

### Results and Discussion

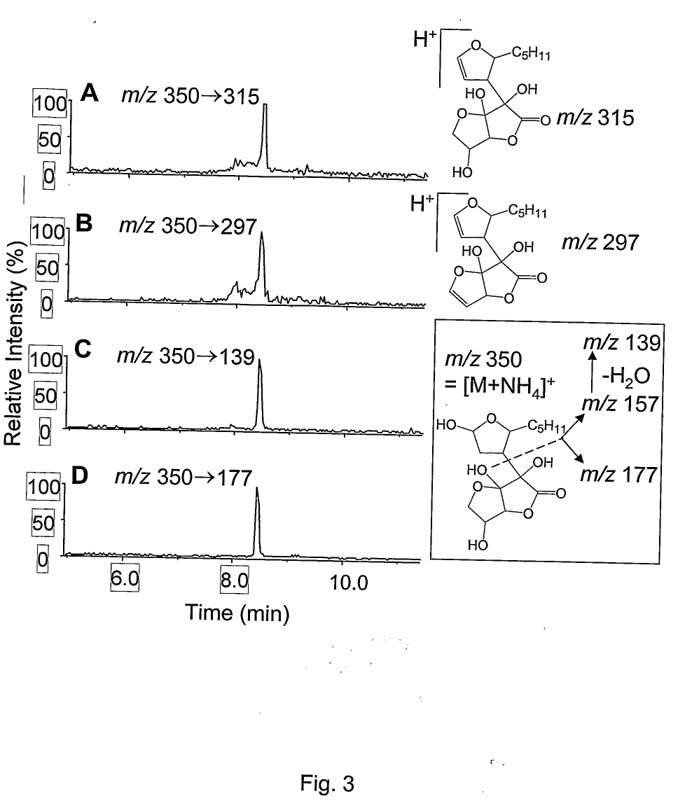

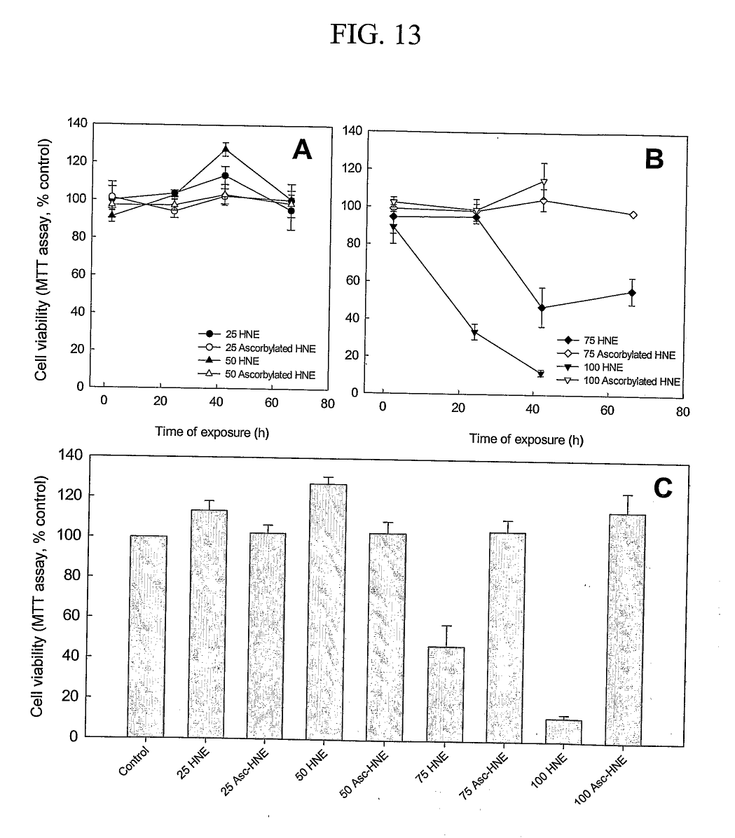

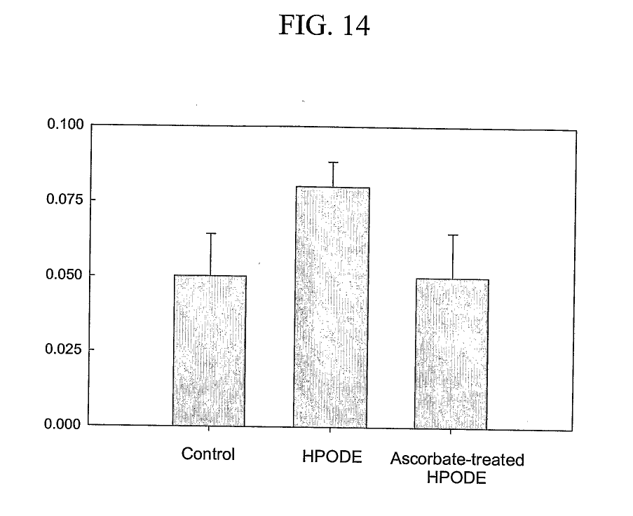

The results presented herein establish that ascorbic acid can induce degradation of lipid hydroperoxides (LOOHs) and subsequently react with the degradation products (LPO products) via Michael chemistry (Scheme 4). Michael-type reactions of electrophilic LPO products with ascorbic acid take place in pseudo-physiological buffer systems (pH 7.4 and 37° C.). The resulting Michael adducts, ascorbylated LPO products, are remarkably stable in aqueous solutions and can be isolated and chemically characterized by mass spectrometry and NMR spectroscopy (FIG. 3). The ascorbylation of the LPO product, HNE, leads to a decrease of HNE's cytotoxicity to HAECs (FIG. 13). Furthermore, these findings indicate that vitamin C-treatment of HPODE results in a decrease of HPODE's capability to induce ICAM-1 expression in HAECs (FIG. 14). These data suggest that ascorbylation is a novel pathway for detoxification of cytotoxic LPO products.

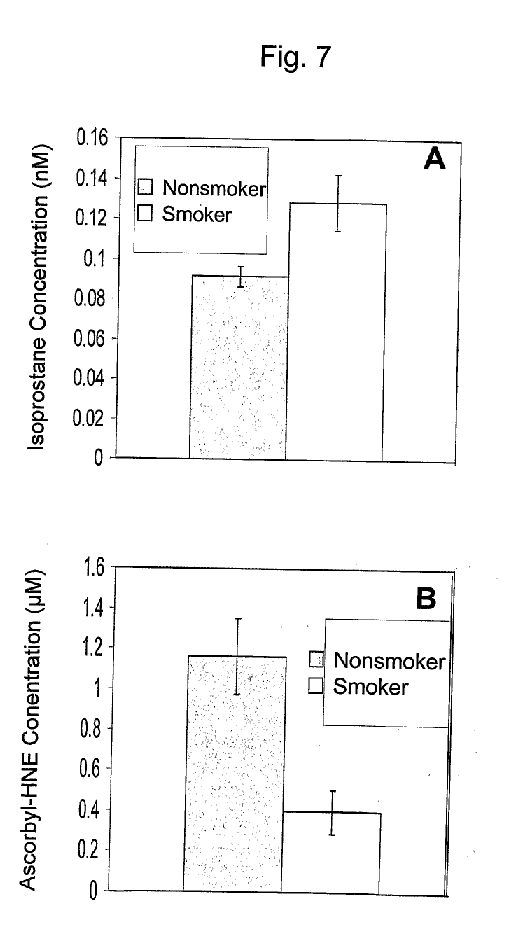

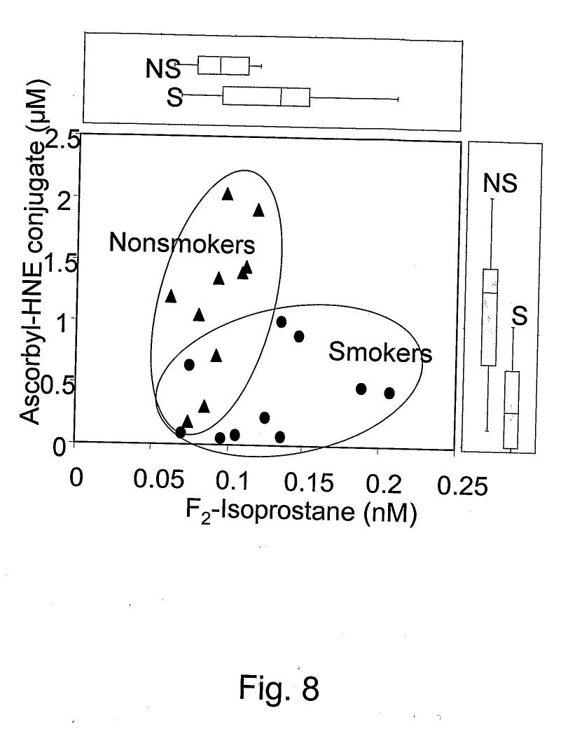

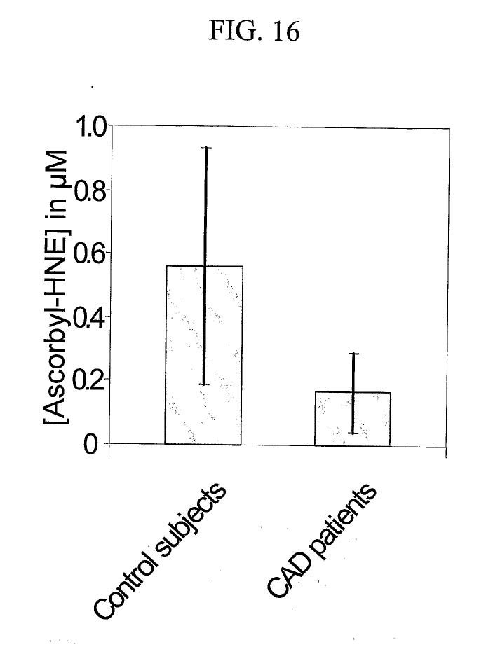

Also demonstrated herein is that ascorbylated LPO products are present in the circulation at levels that far exceed the levels of F₂-isoprostanes normally found in human plasma (FIGS. 7-8). Moreover, the levels of ascorbylated HNE were used to distinguish between smokers and non-smokers (FIG. 7), and between coronary artery disease (CAD) patients and age-matched control subjects (FIG. 16). In summary ascorbic acid is a biological nucleophile (Michael donor) that eliminates LPO products via a biologically relevant pathway.

With reference to Scheme 4, vitamin C can function as a one-electron (1e) donor to HPODE, thereby inducing formation of the alkoxy radical of HPODE. The alkoxy radical then undergoes α,β-carbon-carbon bond cleavage, generating HNE as well as other LPO products. Vitamin C also can function as a Michael (2e) donor and react with HNE and other LPO products, yielding a variety of ascorbyl-LPO product conjugates.

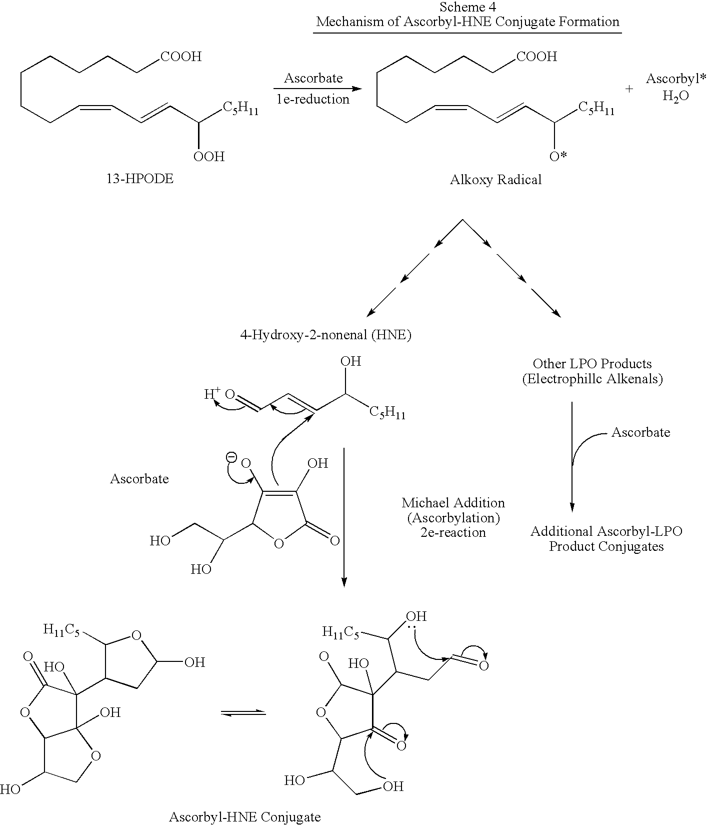

### Ascorbylation of Acrolein

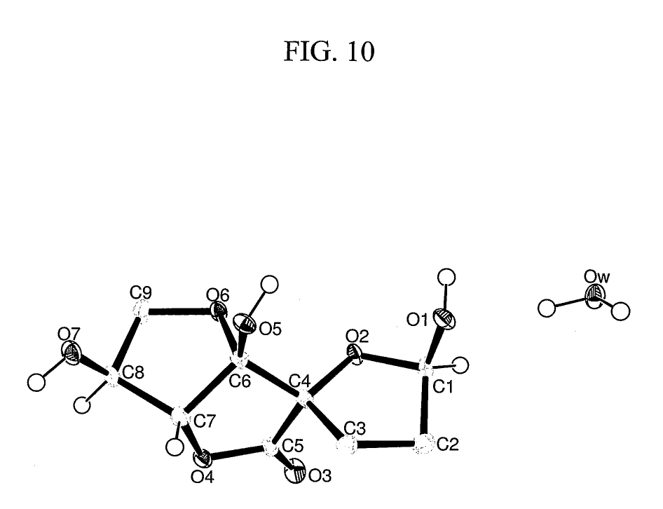

With reference to Scheme 2, acrolein (2-propenal) is a lipid peroxidation product. As demonstrated herein, acrolein can alkylate ascorbic acid via its reactive α,β-unsaturated aldehyde functionality. An aqueous solution of ascorbic acid (1.0 M) was treated with an equimolar amount of acrolein by dropwise addition with stirring at room temperature under nitrogen atmosphere. Following acrolein addition, the solution was left at 4° C. for 5 days, during which period a colorless crystalline material was formed. A well-shaped crystal of dimensions 0.40×0.30×0.30 mm³ was selected and used for X-ray crystallographic analysis. The structure was solved using direct methods as programmed in SHELXS-90 and the solution was refined using the program SHELXL-97, followed by Fourier synthesis, which revealed the positions of the remaining atoms. An Oak Ridge thermal ellipsoid plot (ORTEP) of the final model is given in FIG. 10, with displacement ellipsoids drawn at the 50% probability level.

The mechanism of acrolein ascorbylation was deduced from the crystal structure (FIG. 10) and is shown in Scheme 5. The reaction proceeds in a stereoselective manner and involves Michael addition and two subsequent intramolecular cyclization reactions to give the tricylcic ascorbyl-acrolein conjugate as a single enantiomer.

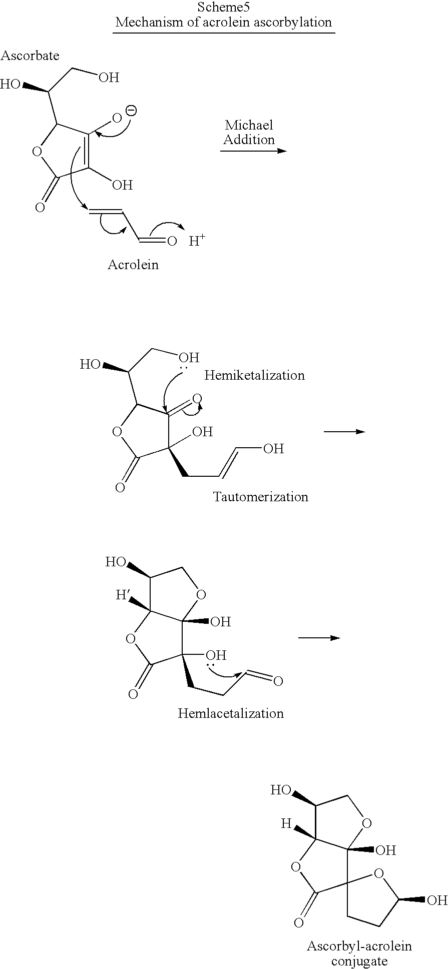

Ascorbylation of 4-hydroxy-2-nonenal and 4-oxo-2-nonenal

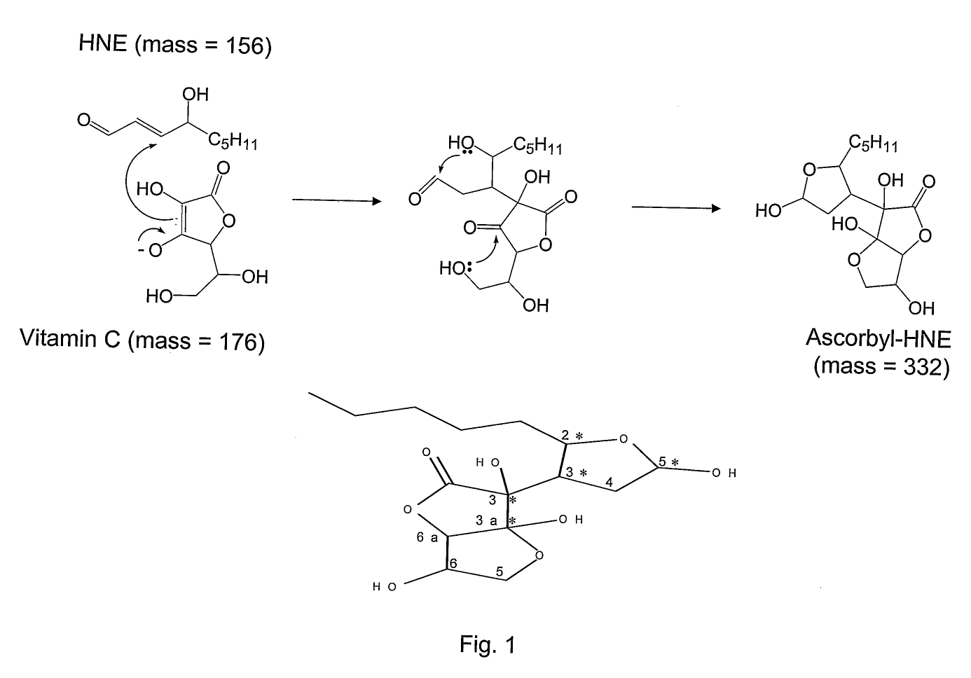

The reaction between ascorbic acid and two LPO products, HNE and 4-oxo-2-nonenal (ONE) also was examined. HNE and ONE were synthesized following literature procedures. In the first set of experiments, HNE was treated with excess ascorbic acid in phosphate buffer pH 7.4 at 37° C. for 2 hours. The reaction product was isolated by semi-preparative HPLC (UV 215 nm) and its structure was elucidated by mass spectrometry and NMR spectroscopy (FIG. 1). The structure of the ascorbyl-HNE conjugate is in agreement with the reaction mechanism of its formation as depicted in Scheme 5. This is an important finding, because it demonstrates that ascorbic acid forms Michael adducts with HNE under pseudo-physiological conditions.

With reference to FIG. 1, asterisks denote newly formed stereo-centers upon ascorbylation of HNE. The ascorbyl-HNE conjugate showed a molecular ion [MH]⁺ with m/z 333.1546 (C₁₅H₂₅O₈⁺ calculates for 333.1549) in the electrospray Q-ToF mass spectrum. Collisional activation of the [MH]⁺ ion yielded two major fragment ions as a result of retro-Michael cleavage of the conjugate: the m/z 139.1108 ion was attributed to [HNE-H₂O]H⁺ (C₉H₁₅O⁺ calculates for 139.1123) and the m/z 177.0420 ion to the protonated ascorbic acid fragment (C₆H₉O₆⁺ calculates for 177.0399). The ¹H (400 MHz) and ¹³C (100 MHz) NMR spectra, recorded in CD₃OD, showed no aldehyde proton signals and only one carbonyl carbon (δ_{C} 174.3), indicating that the initial Michael adduct formed a tricyclic product by hemi-ketal/acetalization of the ascorbyl and HNE moieties (Scheme 5). The facile cleavage of the ascorbyl-HNE linkage in the MS/MS experiment argues against cross ketal/acetalization between the ascorbyl and HNE moieties. The structure of the ascorbyl-HNE conjugate was thus determined to be 3,3α,6-trihydroxy-3-(5-hydroxy 2-pentyl-tetrahydro-furan-3-yl)-tetrahydro-furo[3,2-b]furan-2-one.

Further support for this structure was obtained by ¹H-¹H COSY and ¹H-¹³C HMQC (heteronuclear multiple quantum coherence) experiments. The pentyl moiety was evident from a triplet at δ_{H} 0.95 and a broad multiplet at δ_{H} 1.36. In the HMQC spectrum, the hemi-acetal proton signal at δ_{H} 5.82 (triplet) showed a cross peak with a carbon signal at δ_{C} 102.7; these resonances and others (i.e., δ_{C} 26.5/δ_{H} 1.5-1.6 m, δ_{C} 25.5/δ_{H}1.5-1.6 m, δ_{C} 70/δ_{H}4.05 m) were assigned to positions 5, 4, 3, and 2 of the HNE moiety, respectively, because the oxymethine proton at δ_{H}4.05 interacted with the methylene protons of the pentyl substituent and with the H-3 proton at δ_{H}1.5-1.6 in the COSY spectrum. The ascorbyl moiety showed signals for positions 2 (δ_{C}174.3), 3 (δ_{C}102.7), 3α (δ_{C} 106.7, hemiketal carbon), 5 (δ_{C} 72.5/δ_{H} 3.85 m), 6 (δ_{C} 62.5/δ_{H} 3.6-3.7 m) and 6α (δ_{C} 87.5/δ_{H} 4.45 br s), which were mainly assigned on the basis of correlations observed in the HMQC spectrum.

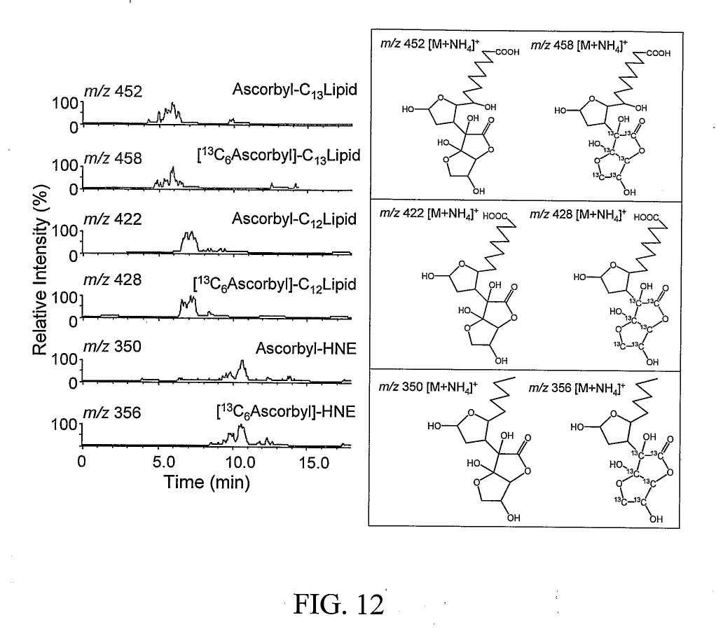

To characterize the ascorbyl-HNE conjugate by mass spectrometry in more detail, HNE was incubated with an isotopomeric mixture of ascorbic acid and [¹³C₆]-ascorbic acid in Chelex-treated phosphate buffer (pH 7.4) at 37° C. Product formation was monitored by liquid chromatography (LC)/electrospray ionization (ESI)/mass spectrometry (US). Because the ascorbyl-HNE conjugate lacks a readily ionizable functional group via protonation or de-protonation, ammonium acetate (10 mM) was added to the LC solvents to detect the conjugates as their ammonium adducts in the positive ion mode. As can be seen in FIG. 12, the pseudo-molecular ions [M+NH₄]⁺ are much more prominently present in the ESI mass spectrum than their corresponding protonated molecules [MH]⁺, demonstrating the increase in detector response due to the presence of ammonium ions. These data show that ascorbyl-HNE conjugates can be detected in reaction mixtures by LC/ESI/MS.

With continued reference to FIG. 12, electrospray mass spectroscopy of ascorbyl-HNE conjugates yielded ions with m/z 350, m/z 333 and m/z 315 representing the ¹²C isotopomer, and ions with m/z 356, m/z 339 and m/z 321 representing the ¹³C₆ isotopomer. MS/MS daughter yielded the m/z 333 [MH]⁺ ion of unlabeled ascorbyl-HNE conjugate, and MS/MS daughter scan of the m/z 339 [MH]⁺ ion of labeled (13C₆) ascorbyl-HNE conjugate.

The data of FIG. 12 were obtained as follows: To a 1.0 ml solution of HNE (5 mM) in 100 mM chelex-treated phosphate buffer (pH 7.4) was added 0.5 mg of unlabeled ascorbic acid and 0.5 mg of isotopically labeled (13C₆) ascorbic acid. The reaction was stirred at 37° C. for 2 h. Conjugates were separated on a C18 column (250×1 mm, 4 μm; Phenomenex, Torrance, Calif.) using a linear solvent gradient starting from 25% B (MeCN) to 85% B in A (10 mM ammonium acetate and 0.1% trifluoroacetic acid in nanopure water) over 45 min at a flow rate of 50 μL/min. The experiments were performed on a PE Sciex API III Plus triple quadrupole mass spectrometer.

Mass fragmentation of the [MH]⁺ and [M+NH₄]⁺ ions of the ascorbyl-HNE conjugate in the MS/MS mode both yielded abundant fragment (daughter) ions with m/z 139 and m/z 177 (FIG. 12). These ions have diagnostic value because they represent the HNE and ascorbyl moieties of the conjugate. These daughter ions can be used for selective and sensitive detection of ascorbylated HNE by LC-MS/MS using multiple reaction monitoring (MRM).

Ascorbylation of the LPO product, 4-oxo-2-nonenal (ONE), in phosphate buffer (pH 7.4, 37° C.) was also studied by LC-MS/MS (data not shown). The ascorbyl-ONE conjugate yielded results similar to the ascorbyl-HNE conjugate, thus supporting the notion that vitamin C acts as a Michael donor for α,β-unsaturated aldehydes under pseudo-physiological conditions.

### Vitamin C-Induced Degradation of HPODEs and Subsequent Ascorbylation of LPO Products

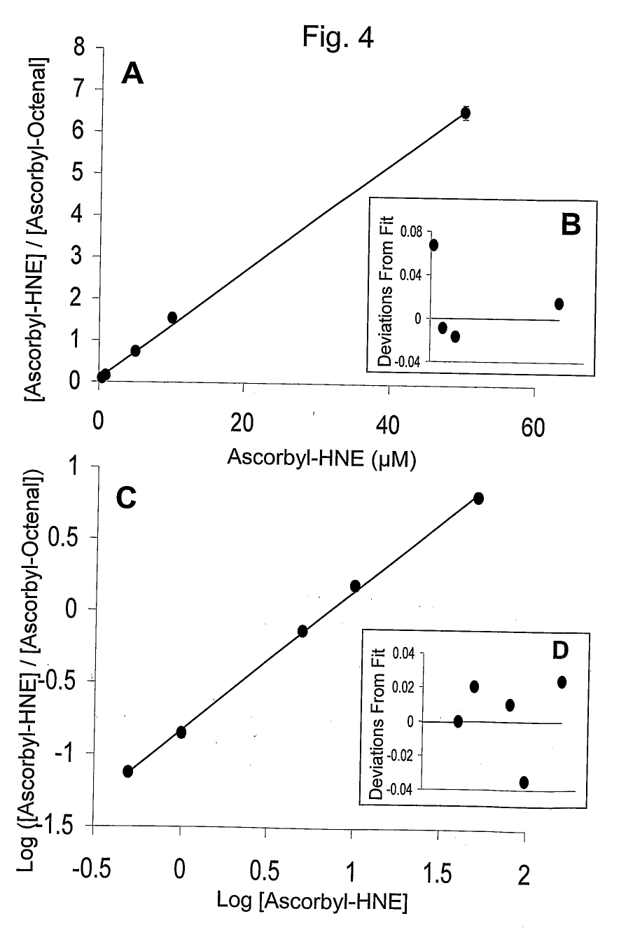

As demonstrated herein, vitamin C initiates the decomposition of HPODEs into electrophilic species and then react with the decomposition products, provided that sufficient vitamin C remains in the reaction solution (Scheme 5). In support of this, HPODEs were decomposed utilizing various concentrations of vitamin C in chelex-treated 100 mM phosphate buffer (pH 7.4) at 37° C. The LC/MS method, utilizing multiple reaction monitoring (MRM), was employed to monitor HNE and ascorbyl-HNE conjugate formation. The results are summarized in FIG. 4. With reference to FIG. 4, each point represents the average of three injections and the error bars indicate mean±SD; panel (A) is graphed on linear scale and panel (C) is graphed on log scale.

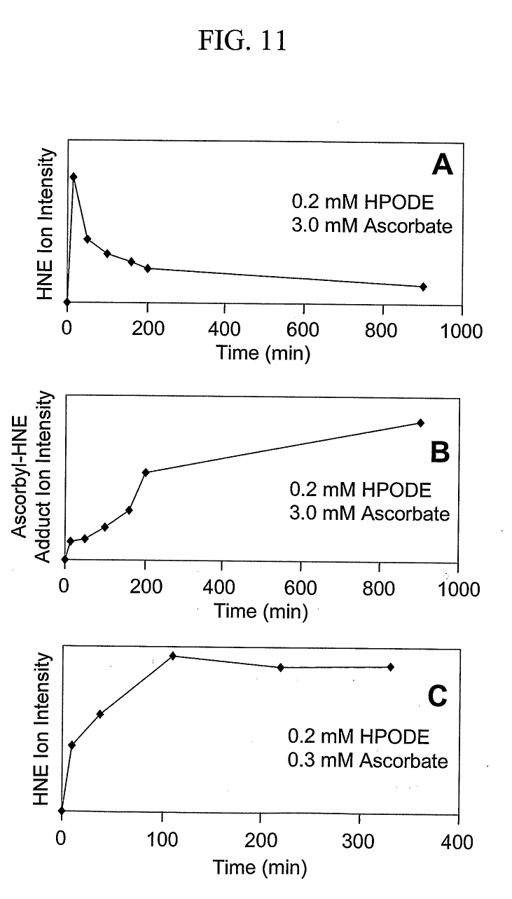

With reference to FIG. 11, panel A illustrates the vitamin C-mediated conversion of HPODE to HNE and subsequent depletion of HNE due to ascorbyl-HNE conjugation; panel B represents concomitant formation of ascorbyl-HNE conjugate; panel C represents vitamin C-mediated conversion of HPODE to HNE without concomitant ascorbyl-HNE conjugate formation due to ascorbate depletion. These data were obtained as follows: To a 1.0 mL solution of HPODE (0.2 mM) in 100 mM chelex-treated phosphate buffer (pH 7.4) was added 0.53 mg (3 mM) of ascorbic acid in the first experiment. The reaction was stirred at 37° C. Aliquots (10 μL) were taken at various time points and analyzed (Panels A and B). The second experiment was carried out with 0.2 mM HPODE in the presence of 0.3 mM ascorbic acid (Panel C). Identical results were obtained for the second experiment when the reactions were carried out in chelex-treated phosphate buffer (pH 7.4) containing 1 mM of the metal ion chelator, diethylenetriaminepentaacetic acid (DTPA), thus confirming that vitamin C is responsible for HPODE decomposition and not traces of iron ions. Using a fifteen-fold excess of vitamin C (3 mM) relative to HPODE, HNE formation peaked at less than 15 min and subsequently decreased over time (FIG. 11, panel A). During the decline in HNE, a concomitant increase in the ascorbyl-HNE conjugate was observed (FIG. 11, panel B). However, when a 1.5-fold molar excess of vitamin C relative to HPODE, a ‘steady state’ of HNE was observed and the ascorbyl-HNE conjugate was not detected, presumably due to depletion of ascorbate as a result of reaction with HPODE and formation of other ascorbyl-LPO product conjugates (FIG. 11, panel C). These results suggest that HNE, produced via vitamin C-mediated decomposition of HPODEs, is readily consumed through Michael-type conjugation with vitamin C. Since vitamin C concentrations far exceed HPODE concentrations in human tissues and in plasma, the data in panels A and B of FIG. 11 are likely to be more relevant to the in vivo situation than the data in panel C where ascorbate is depleted by interaction with HPODE. Nevertheless, the latter situation (panel C) could occur in micro environments of the arterial wall exposed to severe oxidative stress.

13-HPODE was prepared by soybean lipoxygenase-treatment of linoleic acid at pH 8.2. HNE was shown to be absent in this HPODE preparation by LC-MS. For LC-MS/MS analysis of reaction mixtures, formation and disappearance of HNE over time was monitored by the MS/MS transitions, m/z 157 [MH]⁺→m/z 83 and m/z 174 [M+NH₄]⁺→m/z 83. Concomitant appearance of ascorbyl-HNE conjugate was monitored by the MS/MS transitions, m/z 350 [M+NH₄]⁺→m/z 139 [HNE fragment]⁺ and m/z 350 [M+NH₄]⁺→+m/z 177 [Ascorbic acid fragment]⁺.

### Additional Ascorbylated LPO Products as Biomarkers

Vitamin C-induced degradation gives rise to the formation of LPO products other than HNE (Scheme 5). These other LPO products can form conjugates with ascorbic acid via Michael addition in a manner described for HNE above. To demonstrate this, HPODE with an equimolar mixture of ascorbate and ¹³C₆-ascorbate as follows: To a 1.0 ml solution of HPODE (0.2 mM) in 100 mM chelex-treated phosphate buffer (pH 7.4) was added 0.26 mg of unlabeled ascorbic acid and 0.26 mg of isotopically labeled ¹³C₆ ascorbic acid (total ascorbate concentration, 3.0 mM). The reaction was stirred at 37° C. Aliquots (10 μl) were analyzed after 6 hrs. Detection of ascorbyl-LPO product conjugates was initially conducted by identifying compounds showing a ‘mass shift’ of 6 Da, corresponding to the mass difference between the ascorbic acid isoto-pomers. Confirmation of ascorbyl-LPO product conjugates was carried out utilizing MS/MS daughter scanning. The results of the direct LC-MS analysis of the incubation mixture are illustrated in FIG. 12.

With reference to FIG. 12, the total ion chromatogram showed many peaks HPODE degradation experiment. The use of a mixture of ascorbate isotopomers proved extremely useful to distinguish between non-conjugated LPO products and ascorbylated LPO products, because the vitamin C conjugates were readily recognized by chromatographic peaks that showed a mass difference of 6 Da in their mass spectra. Eight products were identified as vitamin C adducts, two of which were identified as ascorbyl conjugates of 13-oxo-9,10-dihydroxy-11-tridecenoic acid and 12-oxo-9-hydroxy-dodecenoic acid (FIG. 12; upper and middle panel, respectively). Without limitation to theory, it is believed that 13-oxo-9,10-dihydroxy-11-tridecenoic acid, an unknown LPO product, is formed via epoxidation and hydrolysis of the known LPO product, 13-oxo-9,11-tridecadienoic acid. The second LPO product, 12-oxo-9-hydroxy-dodecenoic acid, has been described in the literature.

Collectively, the data unequivocally confirm that vitamin C has the ability to degrade LOOHs and form Michael conjugates with the degradation products (LPO products) as summarized in Scheme 5. In addition, the data demonstrate the development of selective and sensitive methods for detection of LPO products and their ascorbyl conjugates.

### Cytotoxicity of HNE and Ascorbylated HNE in Human Aortic Endothelial Cells

FIG. 13 illustrates the results of cytotoxicity studies in human aortic endothelial cells (HAECs) using the MTT assay (described below). These studies demonstrate that ascorbylation of HNE abolishes the cytotoxicity of HNE. As can be seen in FIG. 13 (Panels A and B), HAECs show a progressive decrease in cell viability when exposed to increasing concentrations of HNE from 25 to 100 μM. The observed cytotoxic effect of HNE is relatively moderate, which is likely due to the presence of 20% bovine calf serum in the culture medium of HAECs, causing inactivation of HNE by adduction to serum proteins and other serum constituents. More importantly, ascorbylated HNE was not cytotoxic at any of the concentrations tested (from 25 to 100 μM) in contrast to HNE itself (FIG. 13, panels A and B). FIG. 13, panel C further illustrates the large difference in cytotoxicity between HNE and ascorbylated HNE in HAECs treated with both compounds at 75 or 100 μM for 42 hours. The results of these experiments strongly support the hypothesis that ascorbylation represents a detoxification pathway for HNE and other electrophilic LPO products.

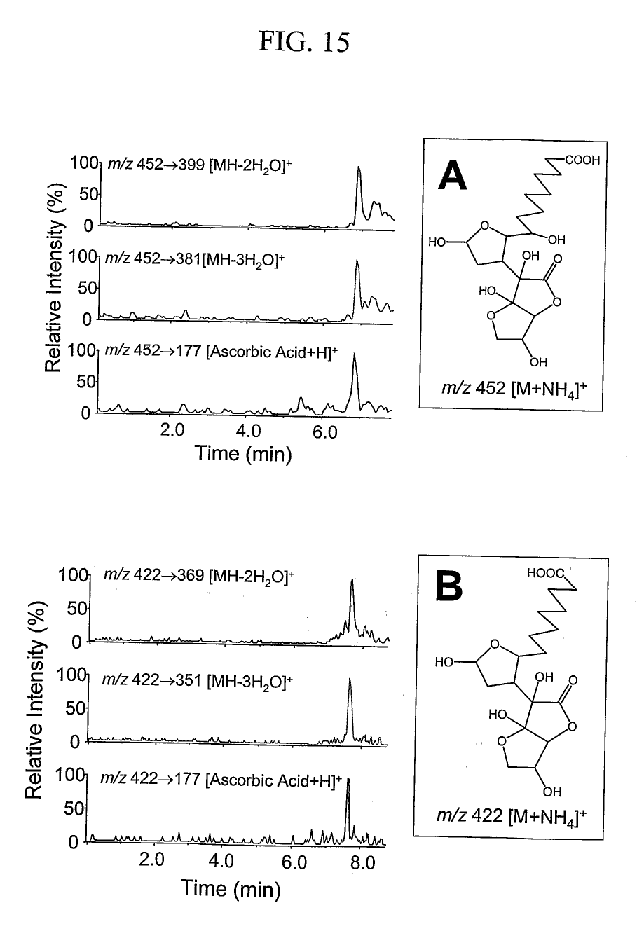

With reference to FIG. 15, Cells were grown to confluence in 96-well plates and exposed to 25, 50, 75 or 100 μM HNE or ascorbylated HNE for up to 66 hours (Panels A and B). Cell viability was assessed by spectrophotometric (590 nm) measurement of the reduction of MTT (3-[4,5-dimethylthiazol-2-yl]-2,5-diphenyltetrazolium bromide) by dehydrogenases in respiring mitochondria, and expressed as a percentage of the control (1.0% ethanol). Panel C shows the effect of HNE and ascorbylated HNE on HAEC viability after 42 hours of exposure. The data are presented as mean of five observations (±SD). Asc-HNE=ascorbylated HNE; numbers in legends refer to μM concentrations.

Modulation of ICAM-1 Expression in HAECs by Oxidized Lipids and their Ascorbyl Conjugates

This example establishes that vitamin C has the ability to abolish HPODE-induced endothelial activation. HAECs were exposed to HPODE or HPODE pretreated with ascorbate. As can be seen from FIG. 14, HPODE treatment caused an increase in ICAM-1 expression, whereas ascorbate-treated HPODE had no effect on ICAM-1 expression compared to the vehicle control. Thus, vitamin C pre-treatment of HPODE leads to inactivation of HPODE with respect to its effect on ICAM-1 expression, which supports the proposition that vitamin C-induced degradation of LOOHs and subsequent ascorbylation of the resultant LPO products represents a pathway for elimination and detoxification of LOOHs and their degradation products (Scheme 2). The increase in ICAM-1 expression in response to HPODE treatment was relatively modest, but it was consistently observed in three separate experiments. As mentioned earlier, there is extensive data in the literature showing that specific LPO products elicit endothelial activation.

With reference to FIG. 14 HPODE was prepared by soybean lipoxygenase treatment of a solution of linoleic acid in phosphate buffer, pH 8.2, with stirring and air bubbling. The product, recovered from the aqueous solution by extraction with diethyl ether, was shown to consist almost exclusively of 13-HPODE by electrospray tandem mass spectrometric comparison with authentic standards of 9- and 13-HPODE (Cayman Chemical Co.) and literature data (Schneider et al. 1997). ‘Ascorbate-treated HPODE’ was prepared as follows: A 1.0 ml-aliquot of an ethanolic HPODE solution (35 mM) was treated with a ten-fold molar excess of ascorbic acid for 12 hrs at 37° C. in 0.1M phosphate buffer, pH 7.4. The solution was acidified, and the mixture of HPODE-derived products was recovered from the aqueous solution by extraction with ethyl acetate. The combined ethyl acetate layers were washed with water to remove traces of ascorbic acid. The residue on evaporation was dissolved in 1.0 ml of ethanol, and the solution termed ‘ascorbate-treated HPODE’. Such a treatment results in the degradation of HPODE and ascorbylation of the resultant degradation products such as HNE (FIG. 11). HAECs were exposed to 35 μM HPODE, 35 μM of ‘ascorbate-treated HPODE’ or 0.1% ethanol (vehicle control) for 24 hours in the presence of 5% bovine calf serum. Activation of HAECs was measured by quantifying ICAM-1 using a commercially available ELISA kit (Zhang and Frei 2001).

The effect of HNE and ascorbylated HNE on endothelial activation also was determined. To this end, BAECs were exposed to 25 μM HNE or ascorbylated HNE for 24 hours in the presence of 20% bovine calf serum, and subsequently ICAM-1 expression was measured by ELISA. Neither HNE nor ascorbylated HNE caused a change of ICAM-1 expression compared to the vehicle control (data not shown). In contrast, HNE was reported to inhibit LPS-induced NFκB activation in human monocytic cells (Herbst et al. 1999; Page et al. 1999) and constitutive ICAM-1 expression in HUVECs (Herbst et al. 1999). This inhibitory effect of HNE on ICAM-1 expression was observed at HNE concentrations as low as 5 μM (Herbst et al. 1999). However, unlike the incubation conditions with 20% bovine calf serum, the experiments by Herbst and colleagues were conducted in a serum-free environment in order to avoid inactivation of HNE by serum proteins (see above).

### Detection of Ascorbylated LPO Products in Human Plasma

The ascorbyl-HNE conjugate was detected in human plasma by LC-MS/MS (FIG. 3). To confirm the identity of the ascorbyl-HNE conjugate in plasma, all four major mass fragment ions of the conjugate were monitored simultaneously during the chromatographic run (FIG. 3, panels A-D). The identity of the conjugate was also confirmed by LC-MS comparison with a synthetic standard.

With reference to FIG. 3, additional LC-MS/MS analysis of a plasma sample from a 38 year-old male demonstrating the presence of the ascorbyl-HNE conjugate was performed. The panels show detection of specific fragment ions, i.e., m/z 315 [M+H—H₂O]⁺ (A), m/z 297 [M+H-2H₂O]⁺ (B), m/z 139 [hydroxynonenal-H₂O+H]⁺ (C) and m/z 177 [ascorbic acid+H]⁺ (D) arising from collisional fragmentation of the quasi-molecular ion with m/z 350 [M+NH₄]⁺ in a multiple-reaction monitoring experiment. The analysis was conducted as follows: Plasma samples (0.2 ml) were acidified with 1N HCl (1.5 ml) and extracted with 3×2 ml volumes of ethyl acetate. The extracts were dried under a stream of nitrogen gas and reconstituted with ethanol-H₂O (1:1) for LC-MS analysis. The LC gradient was from 5% to 75% MeCN in H₂O, containing 10 mM NH₄Ac and 0.1% trifluoroacetic acid, using a 1×250 mm C18 column and a flow rate of 50 μl/min.

To ensure that the ascorbyl-HNE conjugate detected in plasma was not an ex vivo artifact, an aliquot of the plasma sample was incubated with ¹³C₆-ascorbic acid for two hours at room temperature and the sample was analyzed by LC-MS/MS with monitoring of the expected m/z 321, 303, 139 and 183 fragment ions from the pseudo-molecular ion [M+NH₄]⁺ with m/z 356. No 13C₆-ascorbylated HNE was detected, thus confirming in vivo ascorbylation of HNE.

The presence of other ascorbylated LPO products in human plasma also was determined. Ascorbylated ONE (4-oxo-2-nonenal) was detected (data not shown). FIG. 15 shows the LC-MS/MS detection of the two ascorbyl-LPO product conjugates that were also found in the HPODE/ascorbic acid incubation experiment. The analysis results provided in FIG. 15 were obtained using the same procedure as for FIG. 14.

Thus, as demonstrated herein, ascorbylation of LPO products occurs in vivo and that the conjugates can be measured readily by LC-MS/MS.

### Quantification of Ascorbyl-HNE Conjugate in Blood Plasma of Smokers and Cad Patients

Also disclosed herein is a quantitative LC-MS method for analysis of the ascorbyl-HNE conjugate in plasma using ascorbylated 2-octenal as an internal standard. The ascorbyl-(2-octenal) adduct, prepared by treatment of 2-octenal with ascorbic acid and HPLC isolation of the conjugate, was found absent in plasma of three human subjects and thus tentatively considered suitable for use as internal standard. Plasma samples were spiked with a known amount of internal standard and then extracted as described above with reference to FIG. 14. Samples were analyzed by LC-MS/MS using multiple reaction monitoring of at least two diagnostic fragment ions. The concentration of the ascorbyl-HNE conjugate was calculated from a calibration curve, constructed with synthetically prepared ascorbylated HNE and with ascorbyl-octenal adduct as the internal standard. Detector linearity (r²=0.996, n=7) was observed within the concentration range 0.5-100 μM.

### Smokers

Plasma samples from three smokers and three non-smokers, all 20-25 years of age were analyzed as described above. The results are presented in FIGS. 7-9. With reference to FIG. 7, panel B, the smoker group has a significantly lower mean ascorbyl-HNE plasma concentration compared to the non-smoker group (p<0.05).

### CAD Patients

In addition, plasma samples from six patients with angiographically confirmed CAD and seven age-matched control subjects were analyzed by LC-MS/MS using the same method (FIG. 16). The difference between mean plasma levels of both groups is not significant at the p=0.05 level.

The data presented in FIGS. 7 and 16 indicate that smoking-induced or atherosclerosis-associated oxidative stress leads to a decline in the body's capacity to ascorbylate LPO products like HNE. This is a surprising finding, because one would expect an increase in the formation of ascorbylated LPO products in situations of elevated levels of oxidative stress. This also contrasts with other biomarkers, such as F₂-isoprostanes which increase in response to stress. Without being limited to theory, this result may be due to an increase in vitamin C utilization for one-electron pathways in smokers and CAD patients. This would lead to lower vitamin C concentrations at the site of LPO product formation, and, as a result, there would be less vitamin C available for LPO product ascorbylation, a two-electron reaction. This explanation would be in agreement with the data presented in FIG. 11, which show that vitamin C-induced degradation of HPODE and subsequent ascorbylation of the HPODE degradation products (i.e., LPO products) require a large excess of vitamin C relative to HPODE. Furthermore, the higher mean levels of ascorbylated HNE in the healthy control subjects compared to the smokers and CAD patients would lend support to the hypothesis that ascorbylation represents a physiological pathway for detoxification and elimination of harmful LPO products. Note that the plasma levels of ascorbylated LPO products are in the high nanomolar to low micromolar range, which is much higher than the picomolar concentrations of F₂-isoprostanes (HPETE-derived LPO products) normally found in human plasma.

Taken together, these data demonstrate the development of an effective assay for oxidative stress using an LC-MS/MS method for quantification of ascorbyl-HNE conjugate in human plasma because, as demonstrated by the data presented herein, plasma levels of ascorbylated LPO products are modulated by oxidative stress.

### Detection of the Ascorbic Acid-Lipid Peroxidation Conjugates in Human Plasma

This example describes a protocol for the qualitative and quantitative detection of an ascorbic acid lipid peroxidation conjugate in human plasma. The structure of the ascorbyl-HNE adduct, prepared chemically from ascorbic acid and HNE, was determined by NMR spectroscopy and ESI-MS/MS analysis. The presence of the ascorbyl-HNE conjugate in human plasma was established by LC-MS/MS comparison of the synthetic standard with the endogenous conjugate. The retention time of the endogenous conjugate was identical with that of the synthetic standard, which was confirmed by spiking of a plasma sample with the standard. FIG. 2 establishes that the standard and the endogenous conjugate yield virtually identical daughter ion spectra upon collision-induced dissociation (CID) of the [M+NH₄]⁺ ion with m/z 350. These daughter ions arise from loss of ammonia and water molecules (m/z 333 [MH]⁺, m/z 315H—H₂O]⁺, and m/z 297 [MH-2H₂O]⁺) and from cleavage of the carbon-carbon bond between the ascorbyl and HNE moieties (m/z 177 [ascorbic acid+H]⁺ and m/z 139 [HNE+H—H₂O]⁺). The differences in the fragment ion intensities between the standard and the endogenous conjugate are due to the low ion yields and the small number of spectral scans obtained from the endogenous conjugate.

Analysis by LC-MS/MS-MRM, based on the MS/MS fragmentation of the ascorbyl-HNE conjugate, allowed for sensitive and selective detection of the ascorbyl-HNE conjugate in human plasma. FIG. 3 shows the ion currents of four diagnostic fragment ions arising from CID of the [M+NH₄]⁺ ion with m/z 350 in an LC-MRM experiment. With reference to FIG. 3, the panels show detection of specific fragment ions, i.e., m/z 315 [MH—H₂O]⁺ (A), m/z 297 [MH-2H₂O]⁺ (B), m/z 139 [hydroxynonenal-H₂O+H]⁺ (C) and m/z 177 [ascorbic acid+H]⁺ (D) arising from collisional fragmentation of the quasi-molecular ion with m/z 350 [M+NH₄]⁺. The appearance of a single peak matching the retention time of the synthetic adduct indicates that the ascorbyl-HNE conjugate can be detected in human plasma without interference by other plasma constituents, a prerequisite for quantitative analysis of the conjugate in plasma.

A calibration curve allowing for quantitation of the ascorbyl-HNE conjugate in plasma was constructed. Ascorbyl-octenal was used as an internal standard after it had been confirmed that the compound, or an interfering artifact, was not already present in plasma. Varying amounts of the synthetic ascorbyl-HNE adduct were mixed with a fixed amount of internal standard to give 0.5, 1.0, 5.0, 10 and 50 μM concentrations of the analyte and 25 μM of the ascorbyl-octenal adduct. The ratio of their responses was plotted as a function of the ascorbyl-HNE conjugate concentration (FIG. 11, panel A). Linearity was observed over the entire concentration range. While it is unlikely that endogenous ascorbyl-HNE concentrations would exceed the upper concentrations used in the curve, it should be noted that plasma samples are concentrated prior to analysis, thus justifying the inclusion of the upper concentrations used in the calibration curve. It was found that the detection limit of the analysis was 0.1 μM.

A potential problem in using the constructed curve is the systematic increase in variance as the concentration of the ascorbyl-HNE adduct increases as seen by the increasing magnitude of the error bars. Consequently, the higher concentration points are more influential, with respect to curve fitting, than the lower concentration points. A plot of the deviations as a function of the fit illustrates this point (FIG. 11, panel B). The magnitude of the deviation is much higher at the lower concentration points, due to the influence of the increased variance of the higher concentration points. The most straight forward way of circumventing this problem is to construct a log-log plot, thereby more evenly distributing the influence each individual point has on the fit of the curve (FIG. 11, panel C). A plot of the deviations as a function of fit for the log-log plot is shown in FIG. 11, panel D. It can be seen that the deviations are not systematic, indicating that the influence of the higher concentration points has been more evenly distributed.

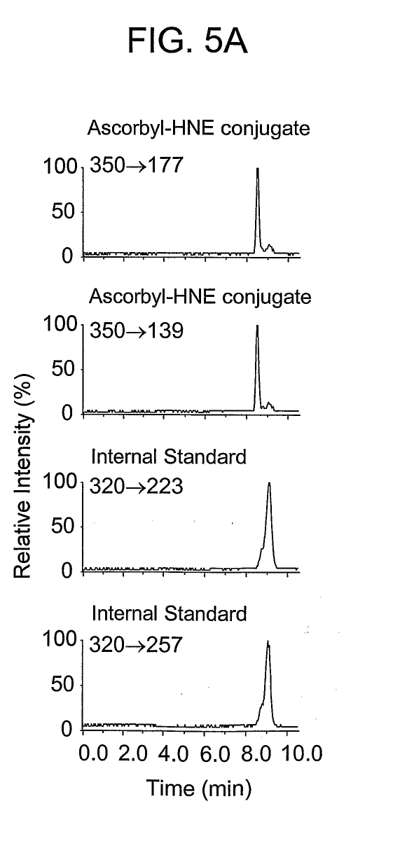

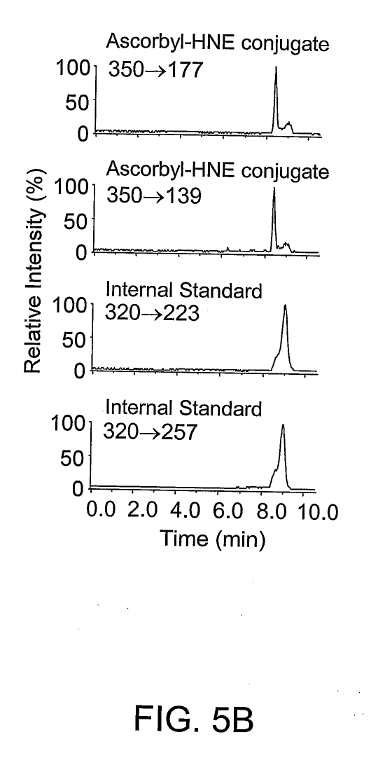

Sample variation due to instrument error and sample preparation was assessed for both groups, nonsmokers and smokers. To test for variation arising from instrumentation, triplicate injections prepared from the same plasma sample were analyzed for the presence of the ascorbyl-HNE conjugate. Using the calibration curve, ascorbyl-HNE conjugate levels were quantitated. FIGS. 5A and 5B give examples of chromatograms showing the endogenous ascorbyl-HNE conjugate and the internal standard for both nonsmokers (FIG. 5A) and smokers (FIG. 5B). With continued reference to FIGS. 5A and 5B, liquid chromatography/tandem mass spectrometry with multiple reaction monitoring was used for the analysis. The upper two panels' peaks arise from retro-Michael fragmentation of the ascorbyl-HNE adduct (retention time 8.4 min). The peaks in the lower two panels are due to fragmentation of the internal standard, ascorbylated 2-octenal (retention time 8.9 min): m/z 320 [M+NH₄]⁺→m/z 257 [MH—H₂O—CO]⁺ and m/z 223 [MH-2H₂O—CO₂]⁺.It was found that the instrument variation was 2.6% RSD for nonsmokers and 3.7% RSD for smokers. Variation arising from sample preparation was examined by analyzing three samples prepared from the same plasma. The total variation due to instrument error and sample preparation was determined to be 3.8% RSD for nonsmokers and 4.0% RSD for smokers.

A problem with current biomarkers of oxidative stress is the formation of ex vivo-artifacts arising from sample instability or sample handling. The presence of the ascorbyl-HNE adduct in plasma is not due to an ex vivo-artifact. Moreover, the stability of the ascorbyl-HNE adduct was confirmed to ensure that its concentration was not fluctuating as a function of time. Specifically, aliquots of a plasma sample were analyzed over a time period of one month. The change in the ascorbyl-HNE conjugate concentration was 5.2% (not significant at the p <0.05 level), demonstrating that the conjugate is stable for at least a month at 4° C.

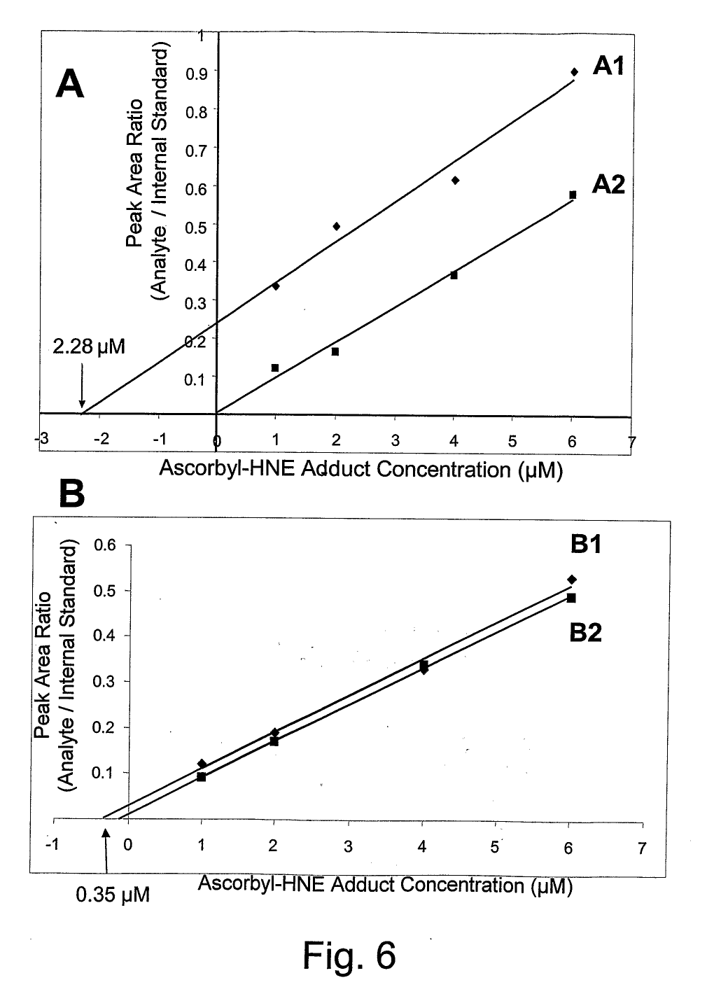

Standard addition experiments were conducted on nonsmoker and smoker plasma to further assess the accuracy and precision of the method developed for determination of the ascorbyl-HNE conjugate concentration in human plasma (FIGS. 6A and 6B). Aliquots of a nonsmoker plasma sample were spiked with synthetic ascorbyl-HNE adduct (1, 2, 4 and 6 μM) and ascorbyl-octenal adduct (25 μM). The samples were extracted as described previously and analyzed by LC/MS/MS-MRM (FIG. 6A). Aliquots of synthetic ascorbyl-HNE adduct and internal standard were run in parallel (FIG. 6B). Extrapolation of curve A1 to y=0 gave an ascorbyl-HNE conjugate concentration of 2.28 μM. A blank plasma sample was analyzed using the calibration curve (FIG. 6, curve A2), and the ascorbyl-HNE conjugate concentration was determined to be 2.68 μM. An analogous experiment using smoker plasma was conducted and the results are shown in FIG. 6, panel B. Extrapolation of curve B1 to y=0 gives an ascorbyl-HNE concentration of 0.35 μM. Analysis of this plasma sample using curve B2 of FIG. 6 gives a concentration of 0.32 μM. The above results demonstrate that the developed method is accurate and reproducible.

### Ascorbyl-Lipid Peroxidation Conjugates as Novel Biomarkers of Oxidative Stress

To demonstrate the utility of the ascorbyl-HNE adduct as a novel biomarker of oxidative stress, plasma samples from twenty subjects, ten smokers and ten nonsmokers, were analyzed utilizing LC/MS/MS-MRM. F₂-isoprostane levels of the plasma samples also were determined. The results are summarized in FIGS. 7A, 7B and Table 1. With reference to FIGS. 7A and 7B, LC-MS/MS injections were done in duplicate and averaged for all subjects. Results in both panels are statistically significant at the p<0.05 level. As expected, the smokers had elevated levels of F₂-isoprostanes (p=0.017). Interestingly, the plasma ascorbyl-HNE conjugate concentrations were inversely related to oxidative stress levels. The mean adduct concentrations in nonsmokers was three times greater, 1.16 d as compared to 0.40 μM for smokers (p=0.002). These results are further illustrated in FIG. 8. It can be seen that smokers have elevated levels of F₂-isoprostanes and lower levels of the ascorbyl-HNE conjugate, relative to nonsmokers. Conversely, nonsmokers had higher levels of the ascorbyl-HNE conjugate and lower levels of F₂-isoprostanes (FIG. 8). Correlations between F2-isoprostane and ascorbyl-HNE levels within groups were not significant (p=0.07 for nonsmokers and p=0.82 for smokers).

TABLE 1
Summary of subject characteristics. All data are reported as mean ± SD.
Table 1. Summary of subject characteristics¹

| Parameter | Nonsmokers (n = 10) | Smokers (n = 10) |
|---|---|---|
| Age (years) | 19.5 ± 2.5 | 21.0 ± 1.7 |
| Height (m) | 1.69 ± 0.15 | 1.76 ± 0.13 |
| Weight (kg) | 63.6 ± 14.4 | 68.0 ± 9.0 |
| BMI (kg/m²) | 22.2 ± 3.2 | 22.0 ± 2.2 |
| Dietary Supplements | none | none |
| Cigarettes/day | 0 | 10.6 ± 3.7 |
| Urinary cotinine (ng/ml) | 27 ± 13 | 2587 ± 1615 |
| Isoprostanes (nM) | 0.092 ± 0.018 | 0.129 ± 0.045 |
| Ascorbyl-HNE (μM) | 1.16 ± 0.65 | 0.40 ± 0.31 |

¹All data are reported as mean ± SD.

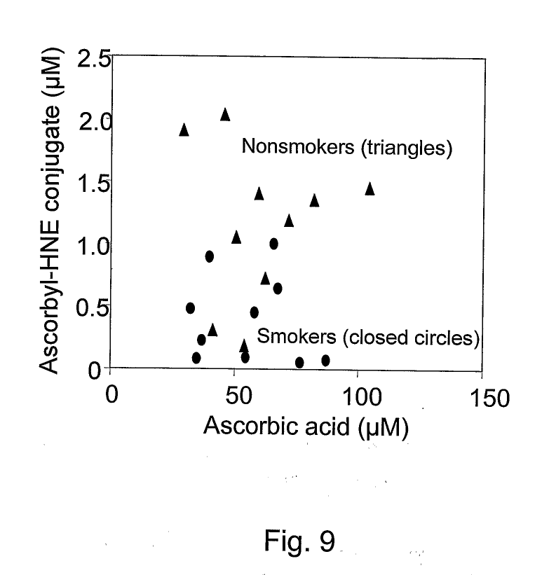

It was of interest to determine if a correlation existed between vitamin C levels and ascorbyl-HNE levels. The average ascorbate levels were 51.5 μM for nonsmokers and 51.6 μM for smokers. FIG. 9 illustrates a plot of plasma ascorbic acid concentration vs. the ascorbyl-HNE conjugate concentration. As can be seen in the figure, no correlation exists in either group (p=0.99 for nonsmokers and p 0.62 for smokers). This observation demonstrates that the plasma levels of the ascorbyl-HNE conjugate do not merely reflect plasma ascorbate levels.

The observed results are somewhat counterintuitive, since it would be reasonable to expect the concentration of the ascorbyl-HNE adduct to increase in response to oxidative stress, as F₂-isoprostanes do. However, under conditions of elevated oxidative stress, intracellular vitamin C may be depleted by one-electron reactions, i.e., antioxidant chemistry, at ‘hotspots’ of LPO processes. As a consequence, insufficient ascorbate may remain available for two-electron ascorbylation of HNE in which ascorbate plays the role of nucleophile. In other words, intracellular ascorbate concentrations may be depleted through oxidative conversion into dehydroascorbic acid (an electrophile rather than a nucleophile), thus explaining the trend observed in the study.

Assuming that LPO products play a beneficial role in defense response to bacterial infections, either by direct interactions of LPO products with the invading microorganism or as inflammatory mediators (like prostaglandins), the body would gain from ‘switching off’ ascorbyl transferase when an optimal inflammatory defense response is desired. The hypothetical control of ascorbyl transferase by oxidative stress is outlined in Scheme 3.

### Ascorbyl-HNE Conjugates in Plasma of Other Mammalian Species

With the aim to identify an animal model for ascorbylation of LPO products, plasma samples from 10 other mammalian species were analyzed for the presence of ascorbylated HNE (Table 2). The results showed a dichotomous distribution with nine species having no detectable plasma levels of ascorbyl-HNE conjugate. A sample of bear plasma contained ascorbylated HNE at about 1 μM, similar to that found in human plasma. Therefore, cultured human cells may best provide an in vitro model for studying LPO product ascorbylation and the factors that regulate it.

TABLE 2
Occurrence of ascorbylated HNE in plasma of mammalian species

| | |
|---|---|
| Species not accumulating the | Species accumulating the |
| ascorbyl-HNE conjugate in | ascorbyl-HNE conjugate in |
| the circulation¹ | plasma at about 1 μM |

| | | |
|---|---|---|
| Goat | Rat | Man |
| Sheep | Pig | Black bear² |
| Cow | Guinea pig | |
| Horse | Dog | |
| | Cat | |

¹Plasma samples (fresh or lyophilized) were obtained from a commercial source or from a local veterinarian. Mouse plasma was not examined.
²A plasma sample was kindly provided by Dr. Wilbert Gamble (Biochemistry &Biophysics, Oregon State University).

There is no reason to assume that the non-accumulating animals of Table 2 have very different levels of ascorbic acid or LPO products compared to humans. Thus, non-accumulating animals should produce similar amounts of ascorbylated HNE compared to humans if in vivo ascorbylation follows normal chemical reaction kinetics. In that case, non-accumulating animals and humans should have very different clearance kinetics regarding the ascorbyl-HNE conjugate. This could be a real possibility if renal excretion is the major route of elimination for the ascorbyl-HNE conjugate, consistent with the conjugate's hydrophilic nature. Another explanation for the observed dichotomy would be provided by the concept of enzymatic ascorbylation of HNE in humans but not in non-accumulating animals.
Spiking of plasma samples with synthetic ascorbyl-HNE adduct

The most direct indication for enzymatic ascorbylation of HNE was obtained from spiking experiments with human plasma. Michael addition of ascorbate to HNE followed by stabilization of the conjugate molecule through hemiacetalization and hemiketalization produces four new asymmetric carbon centers at position 3 of the ascorbyl moiety and positions 2, 3 and 5 of the HNE moiety (see Scheme 4 for reaction pathway and atom numbering). As a consequence, in vitro ascorbylation of HNE yields a mixture of diastereoisomeric conjugates, some of which can be partially resolved on a reversed-phase HPLC column to give broad, split peaks. The chromatographic peak representing the endogenous ascorbyl-HNE conjugate has a sharper and more symmetric shape than the ascorbyl-HNE peak observed after spiking plasma with a synthetic sample of the ascorbyl-HNE adduct (FIG. 11). This is a clear indication that the in vivo formed conjugate is more homogeneous than the synthetic mixture of diastereoisomers, which is best explained by assuming that the in vivo formation of the ascorbyl-HNE conjugate is mediated by an enzyme.

Characterization of Additional Lipid Hydroperoxide Ascorbic Acid Products Derived from Linoleic Acid in Buffer Systems and in Human Plasma.

Linoleic acid is the most abundant polyunsaturated fatty acid in mammalian tissues, and therefore vitamin C conjugation of α,β-unsaturated aldehydes derived from HPODEs produce useful biomarkers. The two positional isomers of HPODE, 13-HPODE and 9-HPODE, will be prepared by treatment of linoleic acid with soybean lipoxygenase following a procedure described by Spiteller et al. (2001). Formation of 13-HPODE is favored at pH values >8 while lowering the pH to 6 causes loss of the enzyme's regiospecificity, yielding a mixture of the two HPODEs that can be resolved by semi-preparative HPLC on silica gel columns using hexane-isopropanol (197:3, v/v) as the mobile phase. Peak fractions (UV detector set at λ=234 nm) will be collected and evaporated. The concentration or yield of both LOOHs will be determined by UV spectrophotometry using the molar absorption coefficient value, ε=23,000 M⁻¹ cm¹ (λₘₐₓ 234 nm). In these studies, it is not important whether LOOHs are formed enzymatically or non-enzymatically, because the asymmetric center formed by lipoxygenase-mediated peroxidation is lost upon conversion of LOOHs into secondary LPO products.

Linoleic acid hydroperoxides (50 mg) will be allowed to decompose in the presence of a 10-fold molar excess of ascorbic acid in phosphate buffer at pH 7.4. Under these conditions, there will be sufficient ascorbic acid remaining for conjugation with the degradation products of the HPODEs. After 5 hours of incubation at 37° C., the reaction mixture will be acidified and the ascorbylated LPO products recovered from the aqueous solution by extraction with ethyl acetate. Individual reaction products will be separated by semi-preparative HPLC on reversed-phase C₁₈ columns and recovered from collected peak fractions by lyophilization. These experiments should yield 0.2-2 mg quantities of material for structure elucidation by mass spectrometry and by 2-dimensional NMR spectroscopy using ¹H—¹H correlation spectroscoy (COSY) and ¹H-13C heteronuclear multiple quantum coherence (HMQC) and heteronuclear multiple bond connectivity HMBC) analysis.

Linoleic acid hydroperoxides will also be allowed to decompose in the presence of an isotopomeric mixture of ascorbate and [¹³C₆]-ascorbate on an analytical scale (5-10 mg) for detailed structural analysis of products by tandem mass spectrometry (MS/MS). Even a homogeneous sample of 13-HPODE likely will produce a large number of products in the presence of ascorbate. Therefore, incubation of HPODE with an isotopomeric ascorbate mixture will aid in the distinction between ascorbyl conjugates and other LPO products as well as in the structural characterization of ascorbylated LPO products. [¹³C₆]-Ascorbate is commercially available from Omicron Biochemicals (South Bend, Ind.).

Characterization of Ascorbylated LPO Products Derived from HPETEs

Arachidonic acid is the second most abundant polyunsaturated fatty acid in mammalian tissues. Despite its lower abundance compared to linoleic acid, arachidonic acid is more readily oxidized and LPO products derived from arachidonic acid also are useful as biomarkers. Furthermore, arachidonic acid is also rapidly released from phospholipids during inflammation by action of phospholipase A₂. Thus, vitamin C-induced degradation of arachidonic acid-derived LOOHs, the HPETEs, and subsequent ascorbylation of the resulting LPO products will be performed. To prepare all HPETE-positional isomers the methyl ester of arachidonic acid will be autoxidized by exposure to air at 37° C. for 48 hours. The resulting mixture of LOOHs will be fractionated by flash chromatography and semi-preparative HPLC on silica gel. This procedure yields milligram amounts of the methyl esters of 15-HPETE, 12-HPETE, 11-HPETE, 9-HPETE, 8-HPETE, and 5-HPETE. The arachidonic acid hydroperoxides will be recovered from the methyl esters by saponification with aqueous lithium hydroxide. The positional isomers will be identified by LC-MS/MS comparison with authentic samples of 5-HPETE, 12-HPETE, and 15-HPETE (Cayman Chemical Co., Ann Arbor, Mich.).

Similar to the experiments described for the linoleic acid hydroperoxides, degradation of individual HPETEs will be induced by a 10-fold molar excess of ascorbic acid in phosphate buffer at pH 7.4. α,β-Unsaturated aldehydes produced under these conditions will react with ascorbic acid to form Michael-type conjugates. The mixture of ascorbyl-LPO product conjugates will be recovered from the acidified aqueous solution by extraction with ethyl acetate. Individual conjugates will be obtained by semi-preparative HPLC on a reversed-phase C₁₈ column and characterized by LC-MS/MS. We choose to perform the ascorbylation experiments with individual HPETE isomers, because this approach will simplify the chemical characterization of the ascorbyl conjugates. Information on HPETE-derived LPO products is available in the literature (Spiteller 2001) while other LPO products can be predicted from known degradation reactions such as 0-scission of HPETE alkoxy radicals, epoxidation of double bonds, and hydrolysis of epoxides.

Identification of Ascorbylated LPO Products its Human Plasma after Addition of HPODEs and HPETEs

Frei and colleagues (Frei, B., Stocker, R., and Ames, B.N. Antioxidant defenses and lipid peroxidation in human blood plasma. Proc Natl Acad Sci USA 1988, 85, 9748-9752) observed that LOOHs are rapidly degraded in human plasma after ex vivo addition. The loss of LOOHs paralleled consumption of ascorbic acid in these experiments, but the fate of the LOOHs was not investigated. Without limitation to theory, it is currently believed that ascorbate-induced degradation and subsequent ascorbylation accounts, at least in part, for the fate of LOOHs added to plasma. Accordingly, HPODEs and HPETEs will be added separately to aliquots of human plasma at 10 μM final concentration and levels of ascorbylated LPO products will be measured for up the three hours of incubation at 37° C. In view of the fact that untreated human plasma contains up to 1-2 μM ascorbylated LPO products (FIG. 4), the amounts of newly formed ascorbylated LPO products will be determined by measuring the difference between untreated and LOOH-treated aliquots of plasma. Quantification will be performed essentially as described for FIG. 14, using ascorbylated 2-octenal as the internal standard.

The ascorbate concentration plays a role in the fate of LOOHs, and therefore LPO product ascorbylation as a function of ascorbate concentration will be monitored, by varying selective removal of endogenous plasma ascorbate using ascorbate oxidase and by addition of ascorbic acid to plasma samples (50-500 μM). Plasma ascorbate concentrations are monitored by HPLC with electrochemical detection as described by Frei and co-workers (Ascorbate is an outstanding antioxidant in human blood plasma. Proc Natl Acad Sci USA 1989, 86, 6377-6381., which is incorporated herein by reference.

Based on the data presented in FIG. 13, low levels of plasma ascorbate will give rise to production of free HNE and other LPO products without subsequent elimination of these degradation products by ascorbylation, due to consumption of ascorbate in one-electron reactions (Scheme 4). The resultant excess of free LPO products may form adducts with plasma proteins through Michael-type reactions, in which lysine and histidine act as nucleophiles. This alternative fate of LOOHs is assessed by quantification of protein-bound lysine-HNE and histidine-HNE adducts as an index for protein adduction to LOOH-derived electrophiles. Protein-bound lysine-HNE and histidine-HNE adducts in plasma will be reduced by NaBH₄-treatment and then hydrolyzed with 6N HCl as described in the literature (Uchida and Stadtman 1994; Requena et al. 1997). The resultant products, 3-(N^{ε}-lysinyl)-4-hydroxynonan-1-ol and 3-(N-histidinyl)-4-hydroxy-nonan-1-ol, will be quantified by LC-MS/MS and expressed as mmol HNB adduct per mol lysine or histidine. In addition, LOOH-derived electrophiles other than HNE will be selected for inclusion in the protein adduction assays based on the outcomes of the HPODE and HPETE studies above.

Thus, structural data for a novel class of ascorbylated LPO products derived from two of the most abundant mammalian polyunsaturated fatty acids, linoleic and arachidonic acid, is generated. Ascorbylation of HPODE-derived electrophiles has resulted in the characterization of ascorbyl conjugates for the LPO products, HNE, ONE, 12-oxo-9-hydroxy-dodecenoic acid, and a partially characterized 13-oxo-tridecenoic acid (see FIG. 14). It is estimated that the proposed studies on vitamin C-induced degradation and conjugation of 9-HPODE and the HPETE positional isomers will yield another 12-20 fully or partially characterized conjugates. Mass fragmentation studies of the ascorbylated LPO products will generate a mass spectral library and provide: the basis for a selective and sensitive LC-MS/MS method for detection and quantification of these conjugates in human plasma. LC-MS/MS will then be used to investigate the fate of HPODEs and HPETEs in human plasma. Furthermore, these studies will determine the extent to which adduction of LPO products to plasma proteins is inhibited by ascorbylation of LPO products at varying ascorbate concentrations.

### Determination of the Relationship Between Oxidative Stress and LPO Product Ascorbylation in Human Subjects

The ‘oxidative modification hypothesis’ of atherosclerosis states that LPO processes contribute to the formation of atherosclerotic lesions in the vascular endothelium. The strong relationship between cigarette smoking and cardiovascular disease is explained, in part, by smoking-induced oxidative stress, which leads to oxidation of lipids and other biomolecules. While the ‘oxidative modification hypothesis’ predicts a beneficial role for vitamin C in the protection against atherosclerosis, there is no satisfactory explanation for how vitamin C interacts with LPO processes. More specifically, the role of vitamin C as a two-electron donor in lipid peroxidation (see Schemes 1 and 4) has not previously been considered as a factor in atherogenesis. However, as demonstrated herein, there is a relationship between oxidative stress and LPO product ascorbylation, a two-electron reaction. Specifically, demonstrated is that the plasma levels of ascorbylated LPO products are modulated by oxidative stress in smokers and coronary artery disease (CAD) patients, by measuring ascorbyl-HNE conjugate and other ascorbylated LPO products in plasma of smokers and CAD patients in two human studies. Thus, as determined in vivo, the interaction of vitamin C with lipid peroxidation is a key factor in atherogenesis. Moreover, based on the plasma levels of ascorbylated LPO products with levels of F₂-isoprostanes, an established marker of in vivo lipid peroxidation, embodiments of the present method using ascorbylated LPO products as novel, unique biomarkers provide a superior system for evaluating oxidative stress. Thus, ascorbylated of LPO products can be used as described herein as in vivo biomarkers of, inter alia, oxidative stress and CAD status.

### Correlation of Additional Biomarkers Via Modulation of Plasma Levels of Ascorbylated LPO Products Following Vitamin C Supplementation in Smokers and Non-Smokers

Twenty-two plasma samples are evaluated using LC-MS/MS measurements of ascorbylated LPO products. These plasma samples were collected in a recently completed randomized cross-over study of antioxidant status in smokers (n=10) and non-smokers (n=12) following vitamin C supplementation. In this study, participants are randomized to 17-day treatments of either ascorbic acid (500 mg; twice daily) or placebo. After a 3-month wash-out period, participants received the alternate treatment. Blood samples (22 subjects×9 collections=198 samples) are collected on days 14, 15, 16 and 17 of each treatment period.

Analysis of the samples as described herein yields ascorbylated LPO products that are relevant to atherosclerosis. These ascorbyl conjugates will be selected for quantification by LC-MS/MS in this study. Plasma samples will be prepared and analyzed in triplicates. The best representative of electrophilic LPO products in terms of abundance, reactivity, and selectivity for discrimination between smokers and non-smokers can thus be selected.

Comparisons between smokers and non-smokers will be carried out using the Student's t-test or the Wilcoxon's rank test in case the data appear not normally distributed. The effect of vitamin C-supplementation on the levels of ascorbylated LPO products in this cross-over study will be analyzed by pairing of the data. Linear regression analysis will be used to examine correlations between levels of ascorbylated LPO products and the other plasma parameters, i.e., ascorbic acid and F₂-isoprostanes. A p value <0.05 will be considered significant.

As described herein, plasma levels of ascorbyl-HNE conjugate are inversely correlated with CAD status (FIG. 16). Plasma vitamin C data should answer the question to which extent reactant concentration plays a role in the ascorbylation reaction. The observed inverse relationship also indicates that, under conditions of increased oxidative stress, a larger proportion of electrophilic LPO products would escape conjugation with ascorbic acid. The ‘excess’ electrophiles could then react with nucleophilic residues in proteins, i.e., lysine and histidine. This would be relevant to the ‘oxidative modification hypothesis’ of atherosclerosis, because oxidative modification of proteins by adduction with LPO products has been related to the conversion of LDL into ox-LDL. Generation of LPO products would normally be more dynamic in the vascular wall than in the circulation, which would argue for analysis of protein-HNE adducts in vascular tissues rather than in plasma. However, in practice this is not possible for obvious reasons, and therefore protein-HNE adducts are measured in plasma proteins as surrogate markers of oxidative damage to cellular proteins as described above.

The up-regulation of cellular adhesion molecules represents a critical step in the initiation and progression of atherosclerosis. Soluble forms of cellular adhesion molecules are released into the circulation upon endothelial activation and can be detected in blood plasma, thus representing an index of cell-surface expression of adhesion molecules. MCP-1 is another inflammatory mediator involved in the recruitment of monocytes by endothelial cells that has been used as a plasma marker for CAD. A decrease in the capacity to ascorbylate electrophilic LPO products may be associated with LPO product-triggered endothelial activation and oxidative modification of LDL. Therefore, levels of ascorbylated LPO products will be correlated with levels of sVCAM-1, sICAM-1 and MCP-1 to examine CAD status and to identify CAD-free individuals that are at risk for developing CAD. Plasma levels of sVCAM-1, sICAM-1 and MCP-1 will be measured by using ELISA kits available from R&D Systems, Minneapolis, Minn.

Smoking is an important risk factor for atherosclerosis. The underlying molecular mechanism of atherogenesis involves overproduction of reactive oxygen species that induce lipid peroxidation. F₂-isoprostanes provide a reliable index of oxidative stress status in vivo and are known to be elevated in smokers. Like F₂-isoprostanes, ascorbylated LPO products are derived from LOOHs, and therefore one would expect a positive correlation between both groups of lipid metabolites. However, the data described herein, for example, FIG. 16, indicate that ascorbylation of LPO products is compromised in smokers and CAD patients, possibly due to increased vitamin C utilization for one-electron pathways in situations of increased oxidative stress (LPO product ascorbylation is a two-electron reaction). This new role for vitamin C has not previously been considered in studies of the effect of vitamin C (supplementation) on cardiovascular diseases.

Low plasma ascorbyl LPO product conjugates is predicted herein for at-risk individuals who are not yet presenting with clinical CAD. It is these individuals that would benefit most from supplementation with vitamin C for the prevention of CAD and possibly other inflammatory diseases that are exacerbated by LPO processes.

Correlation of Adhesion Molecule and MCP-1 Expression with LPO Products and their Ascorbyl Conjugates

Surface expression of adhesion molecules (VCAM-1, ICAM-1 and E-selectin) and MCP-1 expression will be quantified by ELISA performed on HAEC monolayers in flat-bottom 96-well plates. HAECs will be treated for up to 48 hours with LPO products and their ascorbyl conjugates at non-toxic concentrations that will be selected on the basis of the MTT assay results. Ethanol (0.5%) will serve as the vehicle control and treatment with TNFα (10 U/ml) as the positive control. The expression assays using ELISA measurements for VCAM-1, ICAM-1, E-selectin and MCP-1 are known (Zhang, W. J., and Frei, B. Faseb J 2001 15 2423-2432; Cardiovasc Res 2002 55 820-829; and Free Radic Biol Med 2003 34 674-682.

The extent of endothelial activation resulting from exposure of HAECs to LPO products is expected to be a function of the concentration of the free LPO products. Because free LPO products may be inactivated intracellularly by ascorbylation, the concentrations of intracellular ascorbate and ascorbylated LPO products in both the scorbutic and vitamin C-adequate HAECs will be determined. To this end, cellular ascorbate levels by HPLC with electrochemical detection and the levels of ascorbylated LPO products in cell extracts by LC-MS/MS using multiple reaction-monitoring will be measured as we carried out for plasma samples described herein. Ascorbylated LPO products will be prepared as part of studies and used to construct calibration curves for LC-MS/MS quantification.

Electrophilic LPO products may cause damage to cellular proteins by Michael-type adduction, which could lead to increased oxidative stress and endothelial activation. Thus, endothelial activation may depend on a competition between ascorbic acid and nucleophilic amino acid residues (notably lysine and histidine) in proteins for reaction with electrophilic LPO products (i.e., 2-alkenals). Thus, 2-alkenal adduction to cellular proteins will be measured. Protein-bound lysine-alkenal and histidine-alkenal adducts will be reduced with NaBH₄ and then hydrolyzed with 6N HCl. The resultant products, 3-(N′-lysinyl)- and 3-(N-histidinyl)-alkanols, will be quantified by LC-MS/MS and expressed as mmol alkenal adduct per mol lysine or histidine. Adduction of 2-alkenals to proteins can be determined as a competing reaction with ascorbylation for LPO products. Thus, the concentration of ascorbylated LPO products can be correlated with endothelial activation in the assessment of oxidative stress.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A method for identifying a subject at risk of an oxidative stress related disorder, comprising:
obtaining a sample from the subject; and
detecting a concentration of an ascorbic acid-lipid peroxidation product conjugate in the sample.

2. The method of claim 1, wherein the disorder comprises coronary heart disease.

3. The method of claim 1, wherein the disorder comprises atherosclerosis.

4. The method of claim 1, wherein the disorder comprises Alzheimer's disease.

5. The method of claim 1, wherein the disorder comprises an autoimmune disorder.

6. The method of claim 5, wherein the disorder comprises lupus or rheumatoid arthritis.

7. The method of claim 1, wherein the lipid peroxidation product is a linoleic acid derivative or an arachidonic acid derivative.

8. The method of claim 1, wherein the conjugate comprises a 4-hydroxy-2-nonenal residue.

9. The method of claim 1, wherein the conjugate comprises a 13-oxo-9,10-dihydroxy-11-tridecenoic acid moiety or a 12-oxo-9-hydroxy-dodecenoic acid moiety.

10. The method of claim 1, wherein the conjugate has the formula

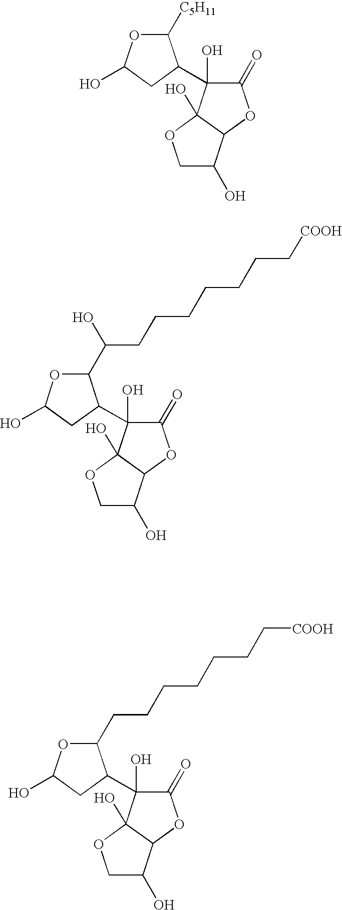

11. The method of claim 3, wherein the lipid peroxidation product is an arachidonic acid derivative.

12. The method of claim 1, wherein the lipid peroxidation product is acrolein.

13. The method of claim 1, wherein detecting comprises liquid chromatography/mass spectrometry.

14. The method of claim 1, wherein the sample is a plasma sample.

15. The method of claim 1, wherein the sample is a urine sample.

16. The method of claim 1, further comprising correlating the concentration of the ascorbic acid-lipid peroxidation product conjugate with sVCAM-1, sICAM-1, E-selectin or MCP-1.

17. A kit for assaying oxidative stress in a subject, comprising an ascorbic acid-lipid peroxidation product conjugate and a reference standard.

18. The kit of claim 17, wherein the lipid peroxidation product is a linoleic acid derivative.

19. The kit of claim 17, wherein the kit comprises a 4-hydroxy-2-nonenal residue.

20. The kit of claim 17, wherein the kit comprises at least one of [chemical structure]
